(19) United States
(12) Patent Application Publication
(10) Pub. No.: US 2026/0215699 A1
(43) Pub. Date: Jul. 30, 2026

(54) MEDICAL DEVICE FOR DETECTING AN ANALYTE IN A BODY FLUID

(71) Applicant: Roche Diabetes Care GmbH, Mannheim (DE)

(72) Inventor: Harald von Campenhausen, Ladenburg (DE)

(21) Appl. No.: 19/578,167

(22) Filed: Mar. 25, 2026

Related U.S. Application Data
(63) Continuation of application No. PCT/EP2024/076605, filed on Sep. 23, 2024.

(30) Foreign Application Priority Data
Sep. 26, 2023 (EP) 23 199 790.9

(51) Int. Cl. A61B 5/0537

(52) U.S. Cl. CPC A61B 5/0537; A61B 2560/0468; A61B 2562/14

(57) ABSTRACT

A medical device for detecting an analyte in a body fluid has an electronics unit with a sensor circuit. An analyte sensor has an insertable portion adapted for at least partially being inserted into a body tissue. The sensor has two or more conductor paths configured for activating the sensor circuit and also has two electrodes each having an electrode conductor path configured for transmitting a sensor current for detecting the analyte. An insertion cannula has the analyte sensor at least partially placed therein. A housing is provided that has an electronics compartment that at least partially receives the electronics unit. The housing has an open channel at least partially surrounding the analyte sensor and the cannula. When the insertion component retracts from the open channel, it triggers electrical contact of the two conductor paths by a connector and this activates the sensor circuit.

### RELATED APPLICATIONS

This application is a continuation of PCT/EP2024/076605, filed Sep. 23, 2024, which claims priority to EP 23 199 790.9, filed Sep. 26, 2023, the entire disclosures of both of which are hereby incorporated herein by reference.

### BACKGROUND

This disclosure relates to a medical device for detecting an analyte in a body fluid, to a method of using a medical device and to a medical device system. The devices and method according to this disclosure may mainly be used for long-term monitoring of an analyte concertation in a body fluid, such as for long-term monitoring of a blood glucose level or of the analyte concentration of one or more other types of analytes in a body fluid. This disclosure may both be applied in the field of home care as well as in the field of professional care, such as in hospitals. Other applications are feasible.

Monitoring certain body functions, more particularly, monitoring one or more concentrations of certain analytes, plays an important role in the prevention and treatment of various diseases. Without restricting further possible applications, this disclosure will be described in the following text with reference to blood-glucose monitoring. However, additionally or alternatively, this disclosure can also be applied to other types of analytes.

Blood glucose monitoring, besides by using optical measurements, specifically may be performed by using electrochemical biosensors. Examples of electrochemical biosensors for measuring glucose, specifically in blood or other body fluids, are known from U.S. Pat. Nos. 5,413,690, 5,762,770, 5,798,031, 6,129,823 or U.S. Publication No. 2005/0013731.

In addition to so-called spot measurements, in which a sample of a bodily fluid is taken from a user in a targeted fashion and examined with respect to the analyte concentration, continuous measurements are increasingly becoming established. Thus, in the recent past, continuous measuring of glucose in the interstitial tissue (also referred to as continuous monitoring, CM), for example, has been established as another important method for managing, monitoring and controlling a diabetes state.

In the process, an active sensor region is applied directly to a measurement site, which is generally arranged in the interstitial tissue, and, for example, converts glucose into electrical charge by using an enzyme (e.g., glucose oxidase, GOD), which charge is related to the glucose concentration and can be used as a measurement variable. Examples of such transcutaneous measurement systems are described in U.S. Pat. No. 6,360,888 B1 or in U.S. Publication No. 2008/0242962A1 .

Hence, current continuous monitoring systems typically are transcutaneous systems or subcutaneous systems, wherein both expressions, in the following, will be used equivalently. This means that an actual sensor or at least a measuring portion of the sensor may be arranged under a skin of the user. However, an evaluation and control part of the system (also referred to as a patch) may be generally situated outside of the body of the user, outside of an human or animal body. In the process, the sensor may be generally applied using an insertion instrument, which is likewise described in U.S. Pat. No. 6,360,888 B1 in an exemplary fashion. Other types of insertion instruments are also known.

The sensor typically comprises a substrate, such as a flat substrate, onto which an electrically conductive pattern of electrodes, conductive traces and contact pads may be applied. In use, the conductive traces typically are isolated by using one or more electrically insulating materials. The electrically insulating material typically further also acts as a protection against humidity and other detrimental substances and, as an example, may comprise one or more cover layers such as resists.

WO 2019/122095 A1 discloses a medical system. The medical system comprises: a housing and a preassembled functional module received in the housing. The pre-assembled functional module comprises an analytical sensor for detecting at least one analyte in a body fluid of a user; an electronics unit electrically connected to the analytical sensor and an insertion component for inserting the analytical sensor into a body tissue of the user. Further, the medical system comprises at least one removable protective cap connected to the housing, covering the preassembled functional module.

EP 2 393 417 A1 describes methods and devices to monitor an analyte in body fluid. Embodiments include continuous or discrete acquisition of analyte related data from a transcutaneously positioned analyte sensor automatically or on demand upon request from a user. A family member of EP 2 393 417 A1 is CN 102307518 B.

U.S. Pat. No. 11,246,519 B2 describes an apparatus for insertion of a medical device in the skin of a subject, as well as methods of inserting medical devices. Embodiments include removing a substantially cylindrical cap from an inserter to expose a substantially cylindrical sleeve, removing a cover from a substantially cylindrical container holding sensor components, and fitting the sensor components into the inserter.

EP 3 641 636 B1 describes embodiments which relate generally to applicators of on-skin sensor assemblies for measuring an analyte in a host, as well as their method of use and manufacture. In some aspects, an applicator for applying an on-skin sensor assembly to a skin of a host is described. The applicator includes an applicator housing, a needle carrier assembly comprising an insertion element configured to insert a sensor of the on-skin sensor assembly into the skin of the host, a holder releasably coupled to the needle carrier assembly and configured to guide the on-skin sensor assembly while coupled to the needle carrier assembly, and a drive assembly configured to drive the insertion element from a proximal starting position to a distal insertion position, and from the distal insertion position to a proximal retraction position.

A miniaturization of continuous glucose monitoring devices may benefit from being able to use a small battery. This in turn may be achieved if during the time between manufacturing and activation of the continuous monitoring device a power consumption can be minimized. In state of the art of integrated of continuous glucose monitoring devices, an electronics unit may generally switch back and forth between a wake-up mode and a low power mode, the latter may also be referred to as sleep mode and a wake-up mode. During the wake-up mode, the electronics unit of the continuous glucose monitoring device may assess if the sensor has been tampered with and/or opened, in which case the electronics unit usually transitions to an activated state in which the device may start to detect analyte concentration and/or establish wired or wireless communication with a remote monitoring device. If no activation is detected, the electronics unit may switch back to the low power mode. This mechanism may be associated with the disadvantage that battery capacity and, as a result, also the size of the battery need to be chosen such that the battery capacity not only supports the power consumption during a shelf life of, e.g., 1-12 months, of the continuous monitoring device but also during a use period of usually 1 to 2 weeks of wear time. Thus, it is desirable to provide a continuous monitoring device where the battery is completely switched off during the time of storage until the continuous monitoring device is used by the user such that much less power capacity is needed. In turn, designing a miniaturized continuous monitoring device would be allowed which is smaller compared to currently used systems. Further, the continuous monitoring device would be, thus, less expensive to manufacture and more user friendly in use, inter alia because smaller sensors are less visible to the outside world and less likely to be felt when, e.g., bumping into or touching the body part with the worn sensor.

Current continuous glucose monitoring devices may have a housing with an electronics compartment with the electronics unit received therein and a sterile sensor compartment for the analyte sensor formed by the insertion cannula, its holder and a sterility cap together with a sealed cylindrical opening of the housing. An insertable sensor part of the analyte sensor may be sealed off from the electronics compartment and from a space within the sterility cap. Thus, there may be an additional challenge to seal off an electronic connecting part of the analyte sensor which may penetrate the housing, specifically the electronics compartment of the housing. Further, the sterility cap may limit the possibility to introduce switching mechanisms into the continuous glucose monitoring device. The concept as outlined above may require that, ex-factory, the insertion cannula and the analyte sensor are already mounted within the housing and connected to the electronic unit.

In alternative continuous glucose monitoring device concepts, ex-factory, the analyte sensor and the insertion cannula are not yet connected to the electronics unit. These concepts usually require two parts, i.e., a sensor and/or cannula comprising part and an electronics unit comprising part which the user connects prior to or after insertion of the sensor into the skin. Given that the industry is moving away from these concepts, they may have lost relevance to the market of new continuous glucose monitoring devices.

The use of the concept of the electronics unit switching back and forth between a sleep mode and a wake-up mode may consume a considerable amount of energy which may necessitate a large battery to ensure that there is enough power to support the continuous glucose monitoring device over an entire period of shelf life and during use after insertion of the analyte sensor into the user's body tissue. At the same time currently available switch-on mechanisms are associated with the above described disadvantages. As a result, there may be a need to provide a medical device which overcomes these shortcomings by reducing a power consumption until activation of the medical device while at the same time providing a continuous glucose monitoring device that readily and reliably wakes-up when the analyte sensor is activated and/or inserted. Further, introduction of further interruptions of the sealed off interior of the housing shall be avoided.

### SUMMARY

This disclosure teaches a medical device for detecting an analyte in a body fluid and a method of using a medical device and a medical device system, which solve at least one of the problems mentioned above. This disclosure teaches a medical device for detecting an analyte in a body fluid, a method of using a medical device and a medical device system which allow a reduced power consumption until activation of the medical device while at the same time providing a reliable activation.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “an element” and “the element” may indicate that several elements such as at least two of the elements may be present in particular embodiments.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “conductor paths,” “electrodes,” and “connector,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended. By way of another specific example, the term “two electrodes” as used in this specification and claims means at least two electrodes or two or more electrodes.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, a medical device for detecting an analyte in a body fluid is disclosed. The medical device comprises:
an analyte sensor having an insertable portion adapted for at least partially being inserted into a body tissue of a user, wherein the analyte sensor is configured for detecting the analyte in the body fluid, wherein the analyte sensor comprises at least two conductor paths configured for activating a sensor circuit, wherein the analyte sensor further comprises at least two electrodes each comprising an electrode conductor path configured for transmitting a sensor current for detecting the analyte;
an insertion component comprising an insertion cannula, wherein the analyte sensor is at least partially placed inside the insertion cannula;
an electronics unit comprising the sensor circuit;
a housing having an electronics compartment with the electronics unit at least partially received therein, wherein the housing further comprises an open channel which at least partially surrounds the analyte sensor and the insertion component; and
a connector element, wherein the insertion component is configured for being retracted from the open channel thereby triggering an electrically contacting of the at least two conductor paths of the analyte sensor by the connector element such that the sensor circuit is activated.

The term “user” as used herein is a broad term and is to be given its ordinary and customary meaning to a person of ordinary skill in the art and is not to be limited to a special or customized meaning. The term exemplarily relates to a person intending to monitor an analyte value, such as a glucose value, in a person's body tissue. In an embodiment, the term specifically may refer, without limitation, to a person using the medical device. For example, the user may be a patient suffering from a disease, such as diabetes. The user may also be referred to as subject or as patient. However, in another embodiment, the person using the medical device is different from the user.

The term “medical device” as used herein is a broad term and is to be given its ordinary and customary meaning to a person of ordinary skill in the art and is not to be limited to a special or customized meaning. The term specifically may refer, without limitation, to an arbitrary element or article being configured for use in the field of medical technology, exemplarily in the field of medical analytics or medical diagnostics. The medical device may be configured for performing a medical function and/or for being used in a medical process, such as in one or more of a therapeutic process, a diagnostic process or another medical process.

The medical device specifically may comprise an assembly of two or more components capable of interacting with each other, such as in order to perform one or more diagnostic and/or therapeutic purposes, such as in order to perform a medical analysis. Specifically, the two or more components may be capable of performing a detection of the analyte in the body fluid and/or of contributing to the detection of the analyte in the body fluid. The medical device generally may also be referred to as a sensor assembly, a sensor system, a sensor kit or a sensor device. Further, the medical device generally may also be referred to as wearable analyte sensor system.

The medical device may be configured to be mounted on a skin site of a body part selected from the group consisting of: an arm, exemplarily an upper arm; a stomach; a shoulder; a back; hip; a leg. Specifically, the body part may be the upper arm. However, also other applications may be feasible.

The medical device may comprise a component which may be configured to stay outside of the body tissue. The component which may be configured to stay outside of the body tissue may specifically be the housing having the electronics compartment with the electronics unit received therein. Further, the medical device, specifically the analyte sensor, may comprise, as outlined above, the insertable portion. The insertable portion may be configured for being inserted into the body tissue of the user.

The medical device may be a disposable medical device. The term “disposable medical device” as used herein is a broad term and is to be given its ordinary and customary meaning to a person of ordinary skill in the art and is not to be limited to a special or customized meaning. The term specifically may refer, without limitation, to an arbitrary medical device configured to be disposed of after use. Thus, one or more materials may specifically be low priced and/or easily recyclable. Specifically, the electronics unit may be a single-use electronics unit. The term “single-use” as used herein is a broad term and is to be given its ordinary and customary meaning to a person of ordinary skill in the art and is not to be limited to a special or customized meaning. The term specifically may refer, without limitation, to a property of an arbitrary element of being configured to be applied only for one time. Thus, after detecting the analyte in the body fluid, the user may remove the electronics units from the body tissue, dispose the electronics unit and may utilize a further, new medical device comprising a further, new electronics unit for another detection of the analyte in the body fluid.

Specifically, the analyte sensor, the insertion cannula, the electronics unit, the housing and the connector element may form a pre-assembled single unit. The term “preassembled” as used herein is a broad term and is to be given its ordinary and customary meaning to a person of ordinary skill in the art and is not to be limited to a special or customized meaning. The term specifically may refer, without limitation, to the fact that an assembly process has already taken place. Thus, the components of the medical device may already be assembled, such as by being mechanically interconnected, thereby being ready for use for the function, such as the medical function, e.g., for the analytical function. The pre-assembling specifically may take place in a factory, thereby rendering the medical device a factory-assembled functional module. Specifically, the medical device may be configured such that the user may not see or manipulate the medical device, preferably before putting the medical device to use or before applying it to the body.

The term “body fluid” (used interchangeably herein with “bodily fluid”) as used herein is a broad term and is to be given its ordinary and customary meaning to a person of ordinary skill in the art and is not to be limited to a special or customized meaning. The term specifically relates to a fluid which typically is present in a body or body tissue of a user or a patient and/or which may be produced by the body of the user or the patient. As an example for body tissue, interstitial tissue may be named. Thus, as an example, the body fluid may be selected from the group consisting of blood and interstitial fluid. However, additionally or alternatively, one or more other types of body fluids may be used, such as saliva, tear fluid, urine or other body fluids. During detection of the analyte, the body fluid may be present within the body or body tissue. Thus, specifically, as will be outlined in further detail below, the analyte sensor may be configured for detecting the analyte in the body tissue.

The term “analyte” as used herein is a broad term and is to be given its ordinary and customary meaning to a person of ordinary skill in the art and is not to be limited to a special or customized meaning. The term specifically relates to an arbitrary element, component or compound which may be present in a body fluid and a presence and/or a concentration of which may be of interest for a user, a patient or medical staff such as for a medical doctor. Particularly, the analyte may be or may comprise an arbitrary chemical substance or chemical compound which may take part in a metabolism of the user or the patient, such as a metabolite. As an example, the analyte may glucose, cholesterol, triglycerides, and/or lactate. Additionally or alternatively, however, other types of analytes may be used and/or any combination of analytes may be determined. However, specifically, the analyte may be glucose. In the following, the medical device may specifically be described with respect to glucose monitoring. The detection of the analyte specifically may be an analyte-specific detection.

The term “detecting” as used herein is a broad term and is to be given its ordinary and customary meaning to a person of ordinary skill in the art and is not to be limited to a special or customized meaning. The term specifically relates to a process of determining a presence and/or a quantity and/or a concentration of an analyte. Thus, the detection may be or may comprise a qualitative detection, simply determining the presence of the analyte or the absence of the analyte, and/or may be or may comprise a quantitative detection, which determines the quantity and/or the concentration of the analyte. As a result of the detection, a signal may be produced which characterizes an outcome of the detection, such as at least one measurement signal. The measurement signal specifically may be or may comprise an electronic signal such as a voltage and/or a current. The measurement signal may be or may comprise an analogue signal and/or may be or may comprise a digital signal.

The term “analyte sensor” as used herein is a broad term and is to be given its ordinary and customary meaning to a person of ordinary skill in the art and is not to be limited to a special or customized meaning. The term specifically may refer, without limitation, to a sensor which is capable of qualitatively or quantitatively detecting a presence and/or a concentration of an analyte.

The analyte sensor may particularly be a transcutaneous sensor. The term “transcutaneous sensor” as used herein is a broad term and is to be given its ordinary and customary meaning to a person of ordinary skill in the art and is not to be limited to a special or customized meaning. The term specifically may refer, without limitation, to an arbitrary sensor which is adapted to be fully or at least partly arranged within a body tissue of a patient or a user. For this purpose, the analyte sensor comprises the insertable portion. The term “insertable portion” as used herein is a broad term and is to be given its ordinary and customary meaning to a person of ordinary skill in the art and is not to be limited to a special or customized meaning. The term specifically may refer, without limitation, to a part or component of an element configured to be insertable into an arbitrary body tissue. In order to further render the analyte sensor to be usable as a transcutaneous sensor, the analyte sensor may fully or partially provide a biocompatible surface, i.e., a surface which, at least during durations of use, do not have any detrimental effects on the user, the patient or the body tissue. Specifically, the insertable portion of the analyte sensor may have a biocompatible surface. As an example, the transcutaneous sensor, specifically the insertable portion, may fully or partially be covered with a biocompatible membrane, such as a polymer membrane or gel membrane which is permeable for the analyte and/or the body fluid and which, on the other hand, retains sensor substances such as one or more analyte detection agents within the sensor and prevents a migration of these substances into the body tissue. Other parts or components of the analyte sensor may stay outside of the body tissue. The other parts may be connectable to an evaluation device such as to the electronics units as will further be described below.

The transcutaneous sensor generally may be dimensioned such that a transcutaneous insertion is feasible, such as by providing a width in a direction perpendicular to an insertion direction of no more than 5 mm, preferably of no more than 2 mm, more preferably of no more than 1.5 mm. The sensor may have a length of less than 50 mm, such as a length of 30 mm or less, e.g., a length of 5 mm to 30 mm. As used herein, the term “length” may refer to a direction parallel to an insertion direction. It shall be noted, however, that other dimensions are feasible.

The analyte sensor may specifically be an electrochemical analyte sensor. The term “electrochemical sensor” as used herein is a broad term and is to be given its ordinary and customary meaning to a person of ordinary skill in the art and is not to be limited to a special or customized meaning. The term specifically may refer, without limitation, to a sensor which is configured to conduct an electrochemical measurement, specifically in order to detect an analyte in a body fluid of a user. The term “electrochemical measurement” may refer to a detection of an electrochemically detectable property of the analyte, such as to an electrochemical detection reaction. Thus, for example, the electrochemical detection reaction may be detected by comparing one or more electrode potentials. The electrochemical sensor specifically may be adapted to and/or may be usable to generate an electrical sensor signal which directly or indirectly indicates the presence and/or the extent of the electrochemical detection reaction, such as a current and/or a voltage. The detection may be analyte-specific. The measurement may be a qualitative and/or a quantitative measurement. Still, other embodiments are feasible.

The term “electrode” as used herein is a broad term and is to be given its ordinary and customary meaning to a person of ordinary skill in the art and is not to be limited to a special or customized meaning. The term specifically may refer, without limitation, to an arbitrary element which is configured to or which is usable to electrically or electrochemically detect an analyte. Specifically, each electrode may comprise a conductive pad or conductive element, such as a metal pad and/or a metal element and/or a pad or element made of a conductive inorganic or organic material such as carbon and/or a conductive polymer. The conductive pad or conductive element may be uncovered and/or may be covered with an additional material, such as a sensor chemical. The at least two electrodes of the analyte sensor may be embodied such that an electrochemical reaction may take place at one or more of the electrodes, such as one or more working electrodes. Thus, the electrodes may be embodied such that an oxidation reaction and/or reduction reaction may take place at one or more of the electrodes. The electrochemical detection reaction may be detected by comparing one or more electrode potentials, such as an electrostatic potential of a working electrode with an electrostatic potential of one or more further electrodes such as a counter electrode or a reference electrode. Generally, the two or more electrodes may be used for one or more of an amperometric, an amperostatic, a potentiometric or a potentiostatic measurement. These types of measurements generally are known to the skilled person in the art of analyte detection, such as from WO 2007/071562 A1 and/or the prior art documents disclosed therein. For potential setups of the electrodes, electrode materials or measurement setups, reference may be made to this document. It shall be noted, however, that other setups, electrode materials or measurement setups may be used within this disclosure. The electrodes generally comprise an electrode conductor path configured for transmitting a sensor current for detecting the analyte. The electrode conductor path in turn may in particular embodiments be connected to the sensor electronics, such as via an electrode contact which connects with a corresponding contact of an electronic component of the sensor electronics. As outlined above, the analyte sensor comprises the at least two conductor paths configured for activating the sensor circuit and, additionally, the analyte sensor comprises the at least two electrodes. The at least two conductor paths may be distinct from the at least two electrodes comprising the electrode conductor paths.

Exemplarily, the analyte sensor may comprise a two-electrode sensor. The two-electrode sensor may comprise precisely two electrodes, such as a working electrode and a further electrode such as a counter electrode, e.g., a working electrode and a combined counter/reference electrode. The term “working electrode” as used herein is a broad term and is to be given its ordinary and customary meaning to a person of ordinary skill in the art and is not to be limited to a special or customized meaning. The term specifically may refer, without limitation, to an electrode being adapted for or being usable for performing a electrochemical detection reaction for detecting an analyte in a body fluid. The working electrode may have an analyte detection agent being sensitive to the analyte to be detected. The working electrode may further comprise a conductive working electrode pad. The conductive working electrode pad may be in contact with the analyte detection agent. Thus, the analyte detection agent may be coated onto the conductive working electrode pad. The analyte detection agent may form an analyte detection agent surface which may be in contact with the body fluid. As an example, the analyte detection agent surface may be an open analyte detection agent surface or may be covered by the above-mentioned membrane which is permeable to the analyte to be detected and/or to the body fluid or a part thereof, such that the analyte may interact with the analyte detection agent. For potential analyte detection agents and/or materials for the conductive working electrode pad, again, reference may be made to WO 2007/071562 A1 and/or the prior art documents disclosed therein. Other embodiments, however, are feasible. The one or more “working electrode pads” specifically may be formed by a dot, line or grid which each can form a coherent area of an electrode material. If more than one dot, line or grid of the electrode material is superimposed, the sensor may provide more than one electrode pad. All electrode pads together may build the working electrode. The sensor may comprise the working electrode with a number of electrode pads in a range from 1 to 50, preferably from 2 to 30, preferably from 5 to 20 electrode pads.

The term “analyte detection agent” as used herein is a broad term and is to be given its ordinary and customary meaning to a person of ordinary skill in the art and is not to be limited to a special or customized meaning. The term specifically may refer, without limitation, to an arbitrary material or a composition of materials adapted to change a detectable property in a presence of an analyte. This property may be an electrochemically detectable property. Specifically, the analyte detection agent may be a highly selective analyte detection agent, which only changes the property if the analyte is present in the body fluid whereas no change occurs if the analyte is not present. The degree or change of the property is dependent on the concentration of the analyte in the body fluid, in order to allow a quantitative detection of the analyte. As an example, the analyte detection agent may comprise an enzyme, such as glucose oxidase and/or glucose dehydrogenase.

The at least two electrodes may further comprise the counter electrode. The term “counter electrode” as used herein is a broad term and is to be given its ordinary and customary meaning to a person of ordinary skill in the art and is not to be limited to a special or customized meaning. The term specifically may refer, without limitation, to an electrode adapted for performing an electrochemical counter reaction and adapted for balancing a current flow required by a detection reaction at a working electrode. Additionally or alternatively the at least two electrodes may further comprise a reference electrode. The reference electrode may have a stable and well-known electrode potential. The electrode potential may preferably be highly stable. The counter electrode and the reference electrode may be one of a common electrode or two separate electrodes. Again, for potential materials usable for the counter electrode and/or the reference electrode, reference may be made to WO 2007/071562 A1 and/or the prior art documents disclosed therein. Other embodiments, however, are feasible.

The electrodes, particularly the working electrode, the counter electrode and/or the reference electrode, may have an identical dimension. The term “dimension” may refer to one or more of a width, a length, a surface area, a shape of the working electrode, the counter electrode and/or the reference electrode. A shape of the electrodes may be determined by a manufacturing process, such as a cutting and/or a printing process. The shape may be rectangular or round. Still, other embodiments are feasible, such as embodiments in which the dimensions of the working electrode and the counter/reference electrodes differ and/or embodiments in which a non-circular shape or a non-rectangular shape is used. The electrodes may be made of a non-corrosive and non-passivating material. With regard to possible electrode materials, reference may be made to the prior art documents cited above.

The analyte sensor may comprise a carrier, specifically a substrate. The term “carrier” as used herein is a broad term and is to be given its ordinary and customary meaning to a person of ordinary skill in the art and is not to be limited to a special or customized meaning. The term specifically may refer, without limitation, to an arbitrary element which is suitable to carry one or more other elements disposed thereon or therein. The term “substrate” as used herein is a broad term and is to be given its ordinary and customary meaning to a person of ordinary skill in the art and is not to be limited to a special or customized meaning. The term specifically may refer, without limitation, to an arbitrary flat element which has a lateral extension exceeding its thickness by at least a factor of 2, at least a factor of 5, at least a factor of 10, or even at least a factor of 20 or more.

The carrier, specifically the substrate, specifically may have an elongated shape, such as a strip-shape and/or a bar-shape. The substrate, as an example, may comprise a shaft, specifically a shaft having an elongate shape. For example, the shaft may have a shape selected from the group consisting of a strip, a needle, a tape. Also other shapes may be feasible.

The carrier, specifically the substrate, may be a flexible carrier or substrate, i.e., a carrier or substrate which may be bent or deformed by forces which usually occur during wearing and insertion into the body tissue, such as forces of 10 N or less. Specifically the carrier or the substrate may be made of or may comprise a deformable material, such as a plastic or malleable material and/or an elastic material. As an example, the carrier or the substrate may be or may comprise a foil, such as a foil made of one or more of a paper material, a cardboard material, a plastic material, a metal material, a ceramic material or a glass material. As an example, the carrier or the substrate may comprise a polyimide foil. The carrier or the substrate specifically may comprise an electrically insulating material, such as an electrically insulating plastic foil.

Specifically, the analyte sensor may be a needle-shaped or a strip-shaped analyte sensor having a flexible substrate and the electrodes disposed thereon. As an example, the analyte sensor may have a total length of 5 mm to 50 mm, e.g., a total length of 7 mm to 30 mm. The term “total length” within the context of this disclosure relates to the overall length of the analyte sensor which means a portion of the analyte sensor which is inserted and the portion of the analyte sensor which may stay outside of the body tissue. The portion of the analyte sensor which is inserted may also be called the in-vivo portion, the portion of the analyte sensor which may stay outside of the body tissue may also be called the ex vivo portion. For example, the in vivo portion may have a length in the range from 3 mm to 12 mm. The analyte sensor may further comprise a biocompatible cover, such as a biocompatible membrane which fully or partially covers the analyte sensor and which prevents the analyte detection agent from migrating into the body tissue and which allows for a diffusion of the body fluid and/or the analyte to the electrodes.

As outlined above, the analyte sensor comprises the at least two conductor paths. Specifically, the analyte sensor may comprise two of the conductor paths. The term “conductor path” as used herein is a broad term and is to be given its ordinary and customary meaning to a person of ordinary skill in the art and is not to be limited to a special or customized meaning. The term specifically relates to an arbitrary electrically conducting element which is suited or configured for establishing an electrical connection to another element. The conductor path specifically may have a shape at least in two dimensions. The conductor path specifically may have an elongated shape, such as a length along a substrate exceeding a width in a plane of the substrate by at least a factor of 5, such as at least a factor of 10, or even at least a factor of 100. For example, the conductor path may comprise a wire or trace. Furthermore, the conductor path may comprise an electrically conductive material. Exemplarily, the electrically conductive material may comprise a metallic material. Thus, as an example, the conductor path may comprise gold. In addition or alternatively, other types of metallic materials may be applied, such as at least one of: Cu, Ni, Ag, Au, Pd, Pt. Again, additionally or alternatively, the conductor path may fully or partially be made of a non-metallic electrically conductive material, such as at least one of: a conductive carbon material, such as graphite, graphene, carbon nanotubes, glassy carbon; an electrically conductive organic material, such as an electrically conductive polymer. Also other kinds of electrically conductive materials may be applied additionally or alternatively. Specifically, the at least two conductor paths may be arranged in a distance to each other. Thus, the at least two conductor paths may not touch each other. The at least two conductor paths may be arranged essentially parallel to each other or may extend essentially parallel to each other. Thereby, the term “essentially parallel” may refer to a property of the at least two conductor paths of being parallel to each other. Exemplarily, the at least two conductor paths may be exactly parallel to each other. However, small deviations may be feasible. Specifically, the at least two conductor paths may be arranged at an angle of +/−20°, preferably of +/−10°, more preferably of +/−5° to each other. Further, the at least two conductor paths may have an identical dimensions. For further details on the expression “dimension” reference to the description above is made.

The at least two conductor paths may respectively be disposed on the carrier or on the substrate. Specifically, the at least two conductor paths may be disposed as a layer on the carrier such as on a surface of the carrier. The at least two conductor paths may respectively be at partially covered by a protection layer. The protection layer may be configured for protecting the at least two conductor paths from environmental influences. The protection layer may specifically be made of an electrically insulating material.

Specifically, each of the at least two conductor paths may comprise a contact area. The term “contact area” as used herein is a broad term and is to be given its ordinary and customary meaning to a person of ordinary skill in the art and is not to be limited to a special or customized meaning. The term specifically relates to an arbitrary area of a conductor path having an open or electrically contactable surface. The contact area may be provided in a dimension or direction parallel to the surface, specifically to a supporting surface, of the carrier or the substrate. The contact area may have an arbitrary shape such as a rectangular shape, a polygonal shape or a round shape. Other shapes are possible. The contact areas may be arranged in a distance to each other, specifically opposite to each other. Thus, a first contact area of a first conductor path may be arranged in a distance to a second contact area of a second conductor path, e.g., to prevent that a contact is established in the absence of a contacting connector. Thus, the first contact area and the second contact area may not touch each other.

Specifically, the contact areas may be located on or disposed on a surface of the analyte sensor, specifically on the surface of the carrier, more specifically on a surface of the substrate of the carrier. Specifically, the contact areas of each of the at least two conductor paths may be at least partially located superficially on and/or near the surface of the carrier. As outlined above, the at least two conductor paths may be at least partially covered by the protection area. The contact areas may be exposed. Specifically, the contact areas may be exposed by laser abrasion. In the contact areas, the protection layer may have a recess or a cutout.

Additionally or alternatively, the contact areas may be elevated relative to a surrounding surface of the analyte sensor, specifically relative to a surrounding surface of the carrier. Such an arrangement may improve the contacting of the at least two conductor paths by the connector element. Specifically, the contact areas may be or may comprise a layer of an electrically conductive material which directly or indirectly may be deposited onto the at least two conductor paths and which may provide an electrically contactable surface. Specifically, the contact areas may respectively comprise a soldered area. Thus, specifically, an access of the at least two conductor paths for electrically connecting the connector element may be increased. Specifically, an even more defined contacting surface may be provided by mounting and/or soldering an electrically conductive material onto the contact areas.

The analyte sensor may be partially received inside the electronics compartment while another part of the analyte sensor may be partially received outside the electronics compartment, such as within the open channel. The at least two conductor paths may be at least partially received outside the electronics compartment. Specifically, the at least two conductor paths may be partially received inside the electronics compartment and may be partially received outside the electronics compartment. Specifically, the contact areas of the at least two conductor paths may be arranged outside of the electronics compartment. More specifically, the contact areas of each of the at least two conductor paths may be arranged within the open channel of the housing. The contact areas may specifically be arranged inside the insertion cannula. Further, the at least two conductor paths may respectively have a contact pad for connecting with one or more electronic components of the electronics unit. Specifically, the at least two conductor paths may be configured for connecting with one or more of the electrical energy reservoir such as the battery, the sensor electronics, a switch such as a relay. The contact pads may be distinct from the contact areas of each of the at least two conductor paths. Thus, the at least two conductor paths may respectively comprise a contact pad and a contact area. The contact pads may respectively be arranged inside the electronics compartment. However, there may also be different means than the contact pads for electrically connecting the at least two conductor paths for connecting with the one or more electronics components of the electronics unit such as one or more of a soldering connection, a bonding connection, a plug, a clamping connection or the like.

The term “insertion component” as used herein is a broad term and is to be given its ordinary and customary meaning to a person of ordinary skill in the art and is not to be limited to a special or customized meaning. The term specifically may refer, without limitation, to an arbitrary element which may be insertable at least partially into the body tissue, particularly in order to deliver or to transfer a further element. The insertion cannula may be configured for supporting the insertion of the analyte sensor or the insertion of a part of the analyte sensor. As outlined above, the insertion component comprises the insertion cannula. The insertion component may further comprise a holder for the insertion cannula. The insertion cannula may be attached to the holder. Specifically, the insertion cannula may be fixedly attached to the holder. The holder may at least partially surround the insertion cannula. Specifically, the insertion cannula may have a first end and an opposing second end. The first end may have a tip or a sharp end for inserting the analyte sensor at least partially into the body tissue. The second end may be attached to the holder.

The term “insertion cannula” as used herein is a broad term and is to be given its ordinary and customary meaning to a person of ordinary skill in the art and is not to be limited to a special or customized meaning. The term specifically may refer, without limitation, to a hollow needle which may be at least partially or completely slotted. The analyte sensor may be received within the insertion cannula, such as within a lumen of the insertion cannula. The insertion cannula may comprise a tip or a sharp end for inserting the analyte sensor at least partially into the body tissue. The insertion cannula, e.g., may comprise at least one cross-section selected from the group consisting of: round, elliptical, U shaped, V shaped. Still, other embodiments are feasible. Specifically, the insertion cannula may be a slotted cannula. Alternatively, the insertion cannula may be a non-slotted cannula. The insertion cannula may be configured to be inserted vertically or at an angle of 90° to 30° relative to the body tissue of the user.

The term “electronics unit” as used herein is a broad term and is to be given its ordinary and customary meaning to a person of ordinary skill in the art and is not to be limited to a special or customized meaning. The term specifically relates to an arbitrary device which is configured for performing an electronic function. Specifically, the electronics unit may have an electronic component. Specifically, the electronics unit may comprise the electronic component for one or more of performing a measurement with the analyte sensor, performing a voltage measurement, performing a current measurement, recording sensor signals, storing measurement signals or measurement data, transmitting sensor signals or measurement data to another device. The electronics unit may specifically be embodied as a transmitter or may comprise a transmitter, for transmitting data. Other embodiments of the electronic components are feasible. These electronic components generally are known in the art of long-term monitoring one or more analytes, such as in from one or more of the above-mentioned prior art documents.

The electronics unit may comprise at least one circuit carrier, preferably a printed circuit board, more preferably a flexible printed circuit board. The term “circuit carrier” as used herein is a broad term and is to be given its ordinary and customary meaning to a person of ordinary skill in the art and is not to be limited to a special or customized meaning. The term specifically relates to an element or a combination of elements which are capable of carrying one or more electronic components and of interconnecting these one or more electronics components, such as interconnecting the one or more electronics components electrically or electronically with each other and/or with one or more contact pads. As an example, the circuit carrier may comprise a base and one or more electrical traces and/or one or more electrical contact pads disposed thereon and/or therein. The base, as an example, may be a flat element having a lateral extension which exceeds its width by at least a factor of 10, more preferably by at least a factor of 100 or even a factor of 1000. Other embodiments are feasible. Rigid materials which may be used for the base may be fiber-enforced plastic materials such as fiber-enforced epoxy materials like glass-fiber-enforced epoxy materials such as FR-4. Other materials may be used. Specifically, the base may be a flexible base, such that the circuit carrier may fully or partially be embodied as a flexible printed circuit board. In this case, as an example, the flexible base may fully or partially be made of one or more flexible plastic materials such as one or more plastic foils or laminate, such as polyimides.

An electronic component may be attached to the circuit carrier. The term “electronic component” may generally refer to an arbitrary element or combination of elements which fulfill an electrical or electronic purpose. Specifically, the electronic component may be or may comprise at least one component selected from the group consisting of an integrated circuit, an amplifier, a resistor, a transistor, a capacitor, a diode or an arbitrary combination thereof. The electronic component specifically may be or may comprise a device capable of controlling the analyte sensor, in order to perform an analytical measurement with the analyte sensor. Specifically, the device may comprise a voltage measurement device and/or a current measurement device. Other setups or embodiments are feasible. The electronic component, as an example, may comprise an application-specific integrated circuit (ASIC).

Therein, the electronic component may directly or indirectly be attached to the circuit carrier. The circuit carrier may be a printed circuit board, particularly a flexible printed circuit board. As an example, the electronic component may directly be attached to the circuit carrier by using one or more of soldering, bonding or electrically conductive adhesive. Thus, the circuit carrier may comprise one or more contact pads, wherein corresponding contacts of the electronic component are electrically connected to the one or more contact pads. Additionally or alternatively, however, the electronic component may indirectly be attached to the circuit carrier, such as via an electronic housing. Thus, the electronic housing may be attached to the circuit carrier. Still, an electrical contact between the electronic component and the circuit carrier may be made, such as via a contact passing through the electronic housing. The electronic housing may fully or partially surround the electronic component. As an example, the electronic housing may comprise a lower electronic housing component attached to the circuit carrier, wherein the electronic devices inserted into the lower electronic housing component on a side opposing the circuit carrier. The electronic housing may further comprise a further electronic housing component, such as an upper electronic housing component, which, in conjunction with the lower electronic housing component, may form an encapsulation which fully or partially surrounds the electronic component. Additionally or alternatively, however, other types of encapsulation of the electronic component may be used, such as encapsulation by using one or more casting and/or potting compounds. Thus, as an example, the lower electronic housing component may be used for receiving the electronic component, wherein the upper shell or protection above the electronic component is created by using a casting and/or potting, such as by using one or more of an epoxy, a thermoplastic polymer, a rather, a silicone, and epoxies or the like. Additionally or alternatively, no electronic housing component may be used at all, such as by directly placing the electronic component onto the circuit carrier.

As outlined above, the electronics unit comprises the sensor circuit. The term “sensor circuit” as used herein is a broad term and is to be given its ordinary and customary meaning to a person of ordinary skill in the art and is not to be limited to a special or customized meaning. The term specifically relates to an arbitrary electrical network with a closed-loop or a closable loop giving a return path for current. The electrical network may be or may comprise an interconnection of electrical components. The sensor circuit may specifically be configured for operating the medical device. Different components of the sensor circuit are described below in more detail. Specifically, the electronics unit may comprise the printed circuit board with the sensor circuit disposed thereon. The sensor circuit may comprise the electronic components as described above.

Specifically, the sensor circuit may comprise an electrical energy reservoir, specifically a battery. The term “battery” as used herein is a broad term and is to be given its ordinary and customary meaning to a person of ordinary skill in the art and is not to be limited to a special or customized meaning. The term specifically relates to an arbitrary source of electric power comprising one or more electrochemical cells with external connections for powering an electrical device. When a battery supplies power, its positive terminal may be referred to as cathode and its negative terminal may be referred to as anode. The battery may specifically be a primary battery. The primary battery may be configured for being used once. The primary battery may also be referred to as single-use or disposable battery. The connector element may be configured for establishing an electrical contact between the electrical energy reservoir and electronic components of the sensor circuit by electrically contacting the at least two conductor paths of the analyte sensor.

As outlined above, the sensor circuit is activated when the insertion component is retracted from the open channel thereby triggering an electrically contacting of the at least two conductor paths of the analyte sensor by the connector element; this way the sensor circuit is activated. In particular, this activation may be triggered by removing the insertion cannula from the open channel. Thus, when the insertion cannula is removed from the open channel the connector element may electrically contact the contact areas of each of the at least two conductor paths and the sensor circuit may be closed. Thereby, electronic components of the sensor circuit may be supplied with power from the electrical energy reservoir. Further, the electronics unit, specifically the electronics components of the electronics unit, may be electrically connected to the analyte sensor, specifically to the at least two electrodes of the analyte sensor. Via this electrical connection, the electronics unit may interact with the analyte sensor for performing an electrochemical measurement. This embodiment may specifically be associated with the advantage over hitherto known systems that it may obviate the need for the medical device to switch back and forth between a wake-up mode and a low power mode. Instead, the activation caused by the removal of the insertion component may establish an electrical connection between an energy source, specifically the electrical energy reservoir, and a sensor circuitry, specifically the sensor circuit, for the first time. This way, a miniaturized medical device equipped with an energy source of reduced capacity and size over currently known systems can be provided. Furthermore, the medical device of this disclosure would be, thus, less expensive to manufacture and more user friendly in use, inter alia because a smaller medical device is less visible to the outside world and less likely to be felt when, e.g., bumping into or touching the body part with the worn medical device.

The connector element (also referred to herein as a “connector”) may be configured for establishing a transient or a permanent electrical contact between the at least two conductor paths of the analyte sensor. In the permanent configuration, an electrical connection may be established between the electrical energy reservoir via the at least two conductor paths which are contacted via the connector element and the powered sensor circuit. In another embodiment, the electrical contact may be transient in which case an electrical connection is established between the electrical energy reservoir via the at least two conductor paths which are contacted via the connector element and a switch such as a relay switch. As a result of the electrical signal, the switch such as the relay switch may establish an electrical circuit between the electrical energy reservoir and the sensor circuit which is maintained even after the electrical connection between the at least two conductor paths via the connector element is interrupted, e.g., when the removal of the insertion component from the open channel has been completed.

The term “housing” as used herein is a broad term and is to be given its ordinary and customary meaning to a person of ordinary skill in the art and is not to be limited to a special or customized meaning. The term specifically relates to an arbitrary element which is adapted to fully or partially surround and/or receive one or more elements in order to provide one or more of a mechanical protection, a mechanical stability, an environmental protection against moisture and/or ambient atmosphere, a shielding against electromagnetic influences or the like. Thus, the housing may simply provide a basis for attachment and/or holding one or more further components or elements. Additionally or alternatively, the housing may provide one or more interior spaces for receiving one or more further components or elements. The housing may specifically be manufactured by injection molding. However, other embodiments are feasible. Exemplarily, the electronics unit may be sealed or potted as will further be described below.

The housing may comprise an upper side and a lower side. The terms “upper side” and “lower side” may refer to two opposing sides of the housing. The terms “upper side” and “lower side” may be considered as description without specifying an order and without excluding a possibility that several kinds of upper sides and lower sides may be applied.

Specifically, the upper side may refer to a distal side of the housing. The term “distal side” as used herein is a broad term and is to be given its ordinary and customary meaning to a person of ordinary skill in the art and is not to be limited to a special or customized meaning. The term specifically may refer, without limitation, to an indication of a position of the side of the housing in relation to a user which is furthermost away from a skin site of the user. Exemplarily, for inserting the analyte sensor, the housing may be brought into contact with the skin site of the user. The distal side may refer to a side being distanced to the skin site of the user.

Specifically, the lower side may refer to a proximal side of the housing. The term “proximal side” as used herein is a broad term and is to be given its ordinary and customary meaning to a person of ordinary skill in the art and is not to be limited to a special or customized meaning. The term specifically may refer, without limitation, to an indication of a position of the side of the housing in relation to a user which is closest to a skin site of the user. Exemplarily, for inserting the analyte sensor, the housing may be brought into contact with the skin site of the user. The proximal side may refer to a side being in close proximity to or even in direct contact with to the skin site of the user.

The term “compartment” as used herein is a broad term and is to be given its ordinary and customary meaning to a person of ordinary skill in the art and is not to be limited to a special or customized meaning. The term specifically relates to an arbitrary subpart of a superior element creating a partially or fully enclosed space that may be usable to contain and/or store objects. The subpart may specifically be completely or at least to a large extent closed such that an interior of the compartment may be isolated from a surrounding environment. Exemplarily, the compartment may be separated from other parts of the superior element by one or more walls. Thus, within the housing, two or more compartments may be comprised which may fully or partially be separated from one another by one or more walls of the housing. Each compartment may comprise a continuous space or lumen configured for receiving one or more objects.

As outlined above, the housing comprises the electronics compartment. The term “electronics compartment” as used herein is a broad term and is to be given its ordinary and customary meaning to a person of ordinary skill in the art and is not to be limited to a special or customized meaning. The term specifically relates to an arbitrary compartment which is configured for receiving an element or a combination of elements which fulfill an electrical or electronic purpose. Specifically, the electronic component may be configured for receiving the circuit carrier as outlined above. The circuit carrier may be fixedly positioned within the electronics compartment of the housing. The insertable portion of the analyte sensor and the detachable lower cap may extend downwardly in the open channel beyond a lower surface of the electronics compartment.

The electronics compartment may comprise at least two housing portions. The at least two housing portions may comprise a lower housing portion and an upper housing portion. The terms “lower housing portion” and “upper housing portion” may be considered as description without specifying an order and without excluding a possibility that several kinds of lower housing portions and upper housing portions may be applied. The upper housing portion and the lower housing portion may be connected via one or more of a form-fit connection, a force-fit connection or a connection may material engagement, more specifically by a connection using an adhesive and/or a bonding. The upper housing portion and the lower housing portion may form an encapsulation for the electronics components of the electronics unit.

The lower housing portion may comprise a lower surface configured for being placed on a user's skin. Specifically, the medical device may comprise an adhesive surface for attachment to the user's skin. The term “adhesive surface” as used herein is a broad term and is to be given its ordinary and customary meaning to a person of ordinary skill in the art and is not to be limited to a special or customized meaning. The term specifically relates to a surface being capable to bind to an object and to resist separation. Exemplarily, the adhesive surface may comprise a plaster or an adhesive strip. The plaster or the adhesive strip may comprise an adhesive material. The adhesive surface may be directly or indirectly attached to the housing. The adhesive surface may be or may be located at a lower surface of the electronics compartment or at the lower side of the housing. The term “lower surface” may specifically refer to a surface of the electronics compartment facing the skin user's skin. The adhesive surface may exemplarily have a shape of a circular ring surrounding the analyte sensor.

As outlined above, the housing comprises the open channel. The term “channel” as used herein is a broad term and is to be given its ordinary and customary meaning to a person of ordinary skill in the art and is not to be limited to a special or customized meaning. The term specifically relates to an arbitrary element which may have an elongate shape and which may provide a free volume or lumen and which enables other elements to pass there through. The channel may specifically be an essentially straight channel. As further used herein, the term “straight” may refer to a continuous extension of the channel in one direction essentially without a bend, angle or curve. The channel may extend along a direction of insertion of the analyte sensor. Further, the channel may extend transverse, specifically essentially perpendicular to a direction of extension of the housing. The open channel may specifically have an upper side opening and an opposing lower side opening. The upper side opening may be located on the upper side of the housing facing away from the skin and the lower side opening may be located on the lower side of the housing facing the skin. The open channel may connect the upper side and the lower side. The insertable portion of the analyte sensor may extend downwardly in the open channel beyond the lower side of the housing.

The open channel may from a compartment, specifically a compartment for at least partially receiving the analyte sensor and the insertion cannula. As outlined above, the open channel at least partially surrounds the analyte sensor and the insertion cannula, specifically circumferentially. The open channel may be formed by an open channel wall. The electronics compartment may be at least partially formed by the open channel wall. Thus, the open channel wall may have a first side facing an interior space of the electronics compartment and an opposing second side facing an interior space of the open channel. The open channel wall may at least partially be designed as a cylindrical ring.

The open channel wall may comprise an opening, wherein the analyte sensor passes through the opening. The analyte sensor may be partially received inside the electronics compartment and may be partially located outside the electronics compartment such as within the open channel. The insertable portion of the analyte sensor may be located outside of the electronics compartment. The opening may specifically be a sealed opening. The term “sealed” may generally refer to a property of an arbitrary element of being completely or at least to a large extent isolated from a surrounding environment. The sealed opening may comprise a sealing element. The term “sealing element” may generally refer to an arbitrary element which is configured to cover one or more elements to be sealed off from environmental influences such as liquid, dust, germs, and moisture. The sealing element may seal the electronics compartment from an outer environment. Exemplarily, the sealing element may comprise a sealing lip. As used herein, the term “sealing lip” may refer to a maximum in a cross-sectional profile of the sealing element, which, when the sealing element thereon is pressed on another surface, is the first part of the sealing element to contact the other surface. The profile itself may be symmetric or asymmetric in shape, wherein an asymmetric profile may be favorable. The sealing element may comprise a sealing material, particularly a deformable sealing material, more preferably an adhesive material. The analyte sensor may pass through the sealed opening.

The medical device may comprise a removable upper cap. Further, the medical device may comprise a removable lower cap. The removable upper cap may be configured for removal after insertion of the insertable portion of the analyte sensor into the body tissue. The removable lower cap may be configured for removal before insertion of the insertable portion of the analyte sensor into the body tissue. At least one of the removable lower cap and the removable upper cap may comprise an hygroscopic material, preferably a desiccant, more preferably activated carbon. The removable upper cap may seal with the upper side opening of the open channel and the removable lower cap may seal with a lower side opening of the open channel.

The term “cap” as used herein is a broad term and is to be given its ordinary and customary meaning to a person of ordinary skill in the art and is not to be limited to a special or customized meaning. The term specifically relates to an arbitrary element which is configured to close or to seal a volume. Specifically, the cap may close or seal an opening of an arbitrary container. The terms “upper cap” and “lower cap” may be considered as description without specifying an order and without excluding a possibility that several kinds of upper caps and lower caps may be applied. The removable lower cap and the removable upper cap may be arranged or located on opposing sides of the housing, specifically of the open channel. Specifically, the removable lower cap may be arranged on the lower side of the housing. The removable lower cap may be removably connected, specifically attached, to at least one of the lower side of the housing and the removable upper cap. Specifically, the removable upper cap may be arranged on the upper side of the housing. The removable upper cap may be removably connected, specifically attached, to at least one of the upper side of the housing and the removable lower cap. The removable upper cap and/or the removable lower cap may exemplarily have an elongate shape and may provide an interior volume.

The term “removable” as used herein is a broad term and is to be given its ordinary and customary meaning to a person of ordinary skill in the art and is not to be limited to a special or customized meaning. The term specifically relates to a property of an element of being removable from an arbitrary object. Thereby, a close bonding or contact or a connection between the element and the object may be disconnected. Generally, the element may be removable in a reversible manner wherein the element may be attachable and detachable from the object or in an irreversible manner wherein the element may not be attachable to the object after detachment. Further details are given below in more detail.

The removable upper cap may specifically be or may comprise the holder for the insertion cannula. The removable lower cap may specifically be a sterility cap. The sterility cap may be configured to provide sterile packaging for the insertable portion of the analyte sensor, such that the insertable portion is sealed against a surrounding environment such as against liquid, moisture, dust and germs. The term “sterility cap” as used herein is a broad term and is to be given its ordinary and customary meaning to a person of ordinary skill in the art and is not to be limited to a special or customized meaning. The term specifically may refer, without limitation, to an element such as a cover which is configured for maintaining a sterile atmosphere in a space fully or partially surrounded by the element. The sterility cap, as an example, may be a rigid sterility cap, e.g., made of a rigid plastic material and/or a metal. The sterility cap, as an example, may have a rotational symmetry about an axis which, as an example, may be identical to a rotational symmetry axis of the protective cap and/or of a rotational symmetry axis of the housing. The sterility cap, as an example, may have an elongated shape, with a length exceeding its diameter or equivalent diameter by at least a factor of 2, more preferably by at least a factor of five. The sterility cap, as an example, may have a length of 5 to 20 mm, e.g., a length of 10 to 15 mm.

Embodiments of a medical analyte sensor device comprising an analyte sensor, a cannula, a lower and upper cap, a lower and upper cover, a sterility cap a sterility compartment and an insertion aid are disclosed in EP3202324A1, EP3727130A1, EP3988014A1 and EP3202323A1 which are hereby incorporated herein by reference.

The removable upper cap and/or the removable lower cap may be reversibly or irreversibly connected to the housing and/or to each other. The removable lower cap, the removable upper cap and the open channel may form a sensor compartment for at least partially receiving the analyte sensor. The analyte sensor may be partially received in the electronics compartment and partially received in the sensor compartment. Specifically, the insertable portion may be at least partially received in the sensor compartment. The sensor compartment may be a sealed compartment, specifically a sterile compartment. The sensor compartment may be configured to provide a sterile packaging for the insertable portion of the analyte sensor. The sensor compartment and the electronics compartment may share a common wall. The common wall may be the open channel wall. The term “sealed compartment” as used herein is a broad term and is to be given its ordinary and customary meaning to a person of ordinary skill in the art and is not to be limited to a special or customized meaning. The term specifically may refer, without limitation, to a compartment being isolated from a surrounding environment such that a transfer of gas, fluids and/or solid elements is completely or at least to a large extent reduced. Specifically, the sensor compartment may be configured to provide a sterile packaging for the insertable portion of the analyte sensor. The term “sterile” as used herein is a broad term and is to be given its ordinary and customary meaning to a person of ordinary skill in the art and is not to be limited to a special or customized meaning. The term specifically may refer, without limitation, to a property of an arbitrary object of being at least to a large extent free from all forms of life and/or other biological agents such as prions, viruses, fungi, bacteria or spore forms and also of ingress of liquid, moisture, and dust. Thus, the sterile object may be treated by a sterilization process that eliminates and/or deactivates the forms of life and/or the other biological agents. The sterilization process may comprise one or more of the following techniques: heating, chemical treatment, irradiation, high pressure, filtration. However, other techniques are feasible. The sterilization process may be conducted within a specified region or area of the object such as a surface of the object.

The sensor compartment may comprise an intermediate component. The intermediate component may formed by the open channel wall. Thus, the intermediate component may refer to a compartment formed by the open channel. The term “intermediate component” as used herein is a broad term and is to be given its ordinary and customary meaning to a person of ordinary skill in the art and is not to be limited to a special or customized meaning. The term specifically may refer, without limitation, to an arbitrary component or compartment between at least two other compartments and/or which may be located in another compartment. Thus, the intermediate component may be located in the sensor compartment and may be sealed from the electronics compartment. The intermediate component may be or may comprise an intermediate compartment and/or, as an example, a sealing ring or a ring-shaped element. Specifically, the electronics compartment may at least partially surround the intermediate component. The intermediate component may be at least partially designed as a cylindrical ring surrounding the insertion cannula. The removable upper cap and the removable lower cap may be separated by the intermediate component and may both be removably connected to the intermediate component.

The removable upper cap and/or the removable lower cap may be reversibly or irreversible connected to the housing and/or to each other. The removable upper cap may be at least partially located on the upper side of the housing and/or may be at least partially located within the open channel. Further, the removable upper cap may be configured for closing and/or sealing the upper opening of the open channel. Optionally, the removable upper cap may pass through the lower opening of the open channel, specifically in order for establishing a connection to the removable lower cap.

The removable upper cap may be removably connected to the housing, specifically to a surface of the housing, via a connection, specifically via at least one of a screwing connection, a bayonet connection. The removable lower cap may be removably connected to the housing, specifically to the surface of the housing, via a connection, specifically via at least one of a screwing connection, a bayonet connection. Specifically, the removable upper cap may be reversibly or irreversibly connected to a surface of the upper side of the housing and/or to the removable lower cap. Specifically, the removable lower cap may be reversibly or irreversible connected to a surface of the lower side of the housing and/or to the removable upper cap.

Specifically, the removable lower cap may be pulled off from the housing and/or from the removable upper cap. Further, specifically, the removable upper cap may be pulled off from the housing and/or from the removable lower cap. Thus, the removable lower cap and/or the removable upper cap may, in a stage connected to the housing, overlap with the housing or vice a versa. Additionally or alternatively, the removable lower cap may overlap with the removable upper cap or vice a versa. The housing specifically may comprise a guiding surface for guiding the removable lower cap or the removable upper cap during pulling off the removable lower cap or the removable upper cap during. Additionally or alternatively, the removable lower cap may comprise a guiding surface for guiding the removable upper cap during pulling off the removable upper cap from the removable lower cap or vice a versa. Thus, the removable upper cap may comprise a guiding surface for guiding removable lower cap during pulling off the removable lower cap from the removable upper cap.

Further, specifically, the removable upper cap may be removably connected to the housing at an upper predetermined breaking point and/or the removable lower cap may be removably connected to the housing at a lower predetermined breaking point. As further used herein, the term “predetermined breaking point” may refer to an arbitrary part of an element being configured to break during mechanical load while other parts of the element remain undamaged. Specifically, the predetermined breaking point may comprise a notch wherein a thickness of the element may be smaller in comparison to other parts of the element. The upper predetermined breaking point and/or the lower predetermined breaking point may specifically be ring-shaped breaking points. The terms “upper breaking point” and “lower breaking point” may be considered as description without specifying an order and without excluding a possibility that several kinds of upper breaking points and lower breaking points may be applied.

As outlined above, the medical device comprises a connector element (or “connector”). The term “connector element” as used herein is a broad term and is to be given its ordinary and customary meaning to a person of ordinary skill in the art and is not to be limited to a special or customized meaning. The term specifically relates to an arbitrary electrically conductive element which is configured for electrically contacting a further electrically conductive element. Specifically, the electrically conductive element may be configured for electrically contacting at least two further electrically conductive elements thereby establishing an electrical connection between the at least two further electrically conductive elements. The connector element may be configured for electrically contacting the contact areas of each of the at least two conductor paths. Thereby, an electrical connection between the at least two conductor paths may be established and the sensor circuit may be closed. Further, an electrical connection between the electrical energy reservoir and electronic components of the sensor circuit may be established. Further, the electronic components of the sensor circuit may be electrically connected to the analyte sensor, specifically to the electrodes of the analyte sensor. The electronics unit may interact with the analyte sensor for performing the electrochemical measurement. This process may refer to an activation of the sensor circuit. Thus, the sensor circuit may be in an active state. To the contrary, the sensor circuit may be in an inactive state when the connector element is arranged in a distance to the contact areas of each of the at least two conductor paths, specifically when there is no electrical connection between the connector element and the contact areas of each of the at least two conductor paths. Thus, the sensor circuit may be open or disconnected. Specifically, the electrical connection between the electrical energy reservoir and electronic components of the sensor circuit may be interrupted or disconnected. Further, there may be no interaction between the electronics unit and the analyte sensor for performing the electrochemical measurement. Specifically, the medical device may be inactive as long as the medical device is stored. Specifically, the medical device may be inactive as long as the medical device is not activated during the mounting of the medical device onto the skin of the user, specifically until the insertion component is not being removed from the open channel.

As outlined above, the insertion component, specifically the insertion cannula and/or the holder of the insertion cannula, is configured for being retracted from the open channel thereby triggering an electrically contacting of the at least two conductor paths of the analyte sensor by the connector element such that the sensor circuit is activated. The term “triggering” used herein is a broad term and is to be given its ordinary and customary meaning to a person of ordinary skill in the art and is not to be limited to a special or customized meaning. The term specifically relates to a process which causes another process to start. Thus, the process of retracting, e.g., removing the insertion component, specifically the insertion cannula and/or the holder of the insertion cannula from the open channel may cause the electrically contacting of the at least two conductor paths of the analyte sensor by the connector element such that the sensor circuit is activated. Specifically, by retracting or removing the insertion component from the open channel, the connector element may electrically contact the at least two conductor paths of the analyte sensor. Thereby, the sensor circuit may be activated.

Before insertion of the insertable portion of the analyte sensor into the body tissue, the sensor circuit may be in the inactive state. After insertion of the insertable portion of the analyte sensor into the body tissue, the sensor circuit may be in the active state. Further, before retraction or removal of the insertion component, specifically of the insertion cannula and/or the holder of the insertion cannula, from the open channel, the sensor circuit may be in the inactive state and during and/or after removal or retraction of the insertion component, specifically of the insertion cannula and/or the holder of the insertion cannula, from the open channel, the sensor circuit may be in the active state.

The connector element may be configured for electrically contacting the at least two conductor paths of the analyte sensor by a movement, a displacement, a relaxation or an expansion of the connector element or a component of the connector element. Before retraction or removal of the insertion component, specifically of the insertion cannula and/or the holder of the insertion cannula, from the open channel, the connector element may be arranged in a distance to the contact areas of each of the at least two conductor paths. During and/or after removal or retraction of the insertion component, specifically of the insertion cannula and/or the holder of the insertion cannula, from the open channel, the connector element may be in electrical contact with the at least two conductor paths, respectively, specifically by the movement, the displacement, the relaxation or the expansion of the connector element. Specifically, the connector element may be in direct contact with the at least two conductor paths, respectively, specifically by the movement, the displacement, the relaxation or the expansion of the connector element. The medical device may comprise different embodiments of the connector elements. Preferred embodiments will be described below in more detail.

Specifically, the connector element may be attached to a surface of the open channel wall. Specifically, the connector element may be fixedly attached to the surface of the open channel wall. As an example, the connector element may have a first end and an opposing second end. The connector element may be attached to the surface of the open channel wall via the first end. The opposing second end and/or an intermediate section of the connector element may be configured for electrically contacting the contact areas of each of the at least two conductor paths. Specifically, the connector element may be attached to the surface of the open channel wall facing an interior space enclosed by the open channel. The connector element may be arranged inside the open channel. Further, the connector element may be arranged fully or at least partially outside the electronics compartment. Before removal or retraction of the insertion component, specifically of the insertion cannula and/or the holder of the insertion cannula, from the open channel, the insertion cannula and/or the holder may be configured as a spacer, specifically as a non-conductive spacer, between the connector element and the at least two conductor paths. The contact areas of each of the at least two conductor paths may be located inside the insertion cannula. The connector element may be placed outside of the insertion cannula. Specifically, the contact areas of each of the at least two conductor paths may be arranged opposite to the connector element, specifically opposite to contact areas of each of the connector element. Specifically, before removal or retraction of the insertion component, specifically of the insertion cannula and/or the holder of the insertion cannula, from the open channel, the connector element may be compressed by or tensioned by an outer surface of the insertion cannula. Thus, before removal or retraction of the insertion component, specifically of the insertion cannula and/or the holder of the insertion cannula, from the open channel, the connector element may be in direct contact with the outer surface of the insertion cannula and/or the holder, specifically via the second end and/or the intermediate section of the connector element. By retraction or removal of the insertion component, specifically of the insertion cannula and/or the holder of the insertion cannula, the connector element may be moved, displaced, relaxed or expanded such that the electrical connection between the contact areas of each of the at least two conductor paths and the connector element is established.

The connector element may specifically be a contact spring element (“contact spring”), specifically a bent contact spring element (“bent contact spring”), specifically a leaf contact spring element (“leaf contact spring”) or a conductive elastomeric element (“conductive elastomer”). Before retraction or removal of the insertion component, specifically of the insertion cannula and/or the holder of the insertion cannula, from the open channel, the connector element may be in direct contact with a surface of the insertion component, specifically of the insertion cannula and/or the holder of the insertion cannula. During and/or after removal or retraction of the insertion component, specifically of the insertion cannula and/or the holder of the insertion cannula, from the open channel, the connector element may electrically contact the at least two conductor paths. Specifically, the connector element may be pressed against the contact areas of each of the at least two conductor paths.

The term “contact spring element” (or “contact spring”) as used herein is a broad term and is to be given its ordinary and customary meaning to a person of ordinary skill in the art and is not to be limited to a special or customized meaning. The term specifically relates to an arbitrary contact element whereby a function of closing an electric circuit is achieved by probing by a spring-supported element. The contact spring element may have a contact surface configured for contacting a surface of another element. The contact spring element or at least a portion of the contact spring element may be configured for storing mechanical energy through elastic deformation and for, thus, maintaining a contact with the other element. As outlined above, the contact spring element may specifically be a bent contact spring element. The term “bent contact spring element” (or “bent contact spring”) as used herein is a broad term and is to be given its ordinary and customary meaning to a person of ordinary skill in the art and is not to be limited to a special or customized meaning. The term specifically relates to an arbitrary contact element whereby a shape of the bent contact spring element differs from an essentially straight shape of the contact spring element. Specifically, the contact spring element may extend along at least one of a segment of a circle, a segment of an oval, a segment of an ellipse, a segment of a super ellipse. The bent contact spring element may have a first end and an opposing second end. The bent contact spring element may be attached to the open channel wall via the first end and the second end may protrude into the interior space of the open channel. Specifically, the second end of the bent contact spring element and/or an intermediate section of the bent contact spring element may be in direct contact with the insertion component, specifically of the insertion cannula and/or the holder of the insertion cannula, before retraction or removal of the insertion component, specifically of the insertion cannula and/or the holder of the insertion cannula, from the open channel. Before removal or retraction of the insertion component, specifically of the insertion cannula and/or the holder of the insertion cannula, from the open channel, the contact spring element may be in a tensioned state and the contact spring element may be in direct contact with the surface of the insertion component, specifically of the insertion cannula and/or the holder of the insertion cannula. Specifically, the contact spring element may be pressed against the surface of the insertion component, specifically of the insertion cannula and/or the holder of the insertion cannula. During and/or after removal or retraction of the insertion component, specifically of the insertion cannula and/or the holder of the insertion cannula, from the open channel, the contact spring element may be in a further tensioned state or in a relaxed state and the contact spring element may electrically contact the at least two conductor paths, specifically the contact areas of each of the at least two conductor paths. Thereby, the contact spring element may be pressed against the contact areas of each of the at least two conductor paths. The contact spring element may specifically be made of an electrically conductive material. The contact spring element may be made of any suitable material such as a plastic, thermo-plastic polymer or a metal. Also other materials may be possible.

The term “conductive elastomeric element” (also referred to as “conductive elastomer”) as used herein is a broad term and is to be given its ordinary and customary meaning to a person of ordinary skill in the art and is not to be limited to a special or customized meaning. The term specifically relates to an arbitrary elastomeric element having electrically conductive properties. The conductive elastomeric element may have an elongate shape. Specifically, the conductive elastomeric element may have an essentially straight shape. The conductive elastomeric element may have a first end and an opposing second end. The conductive elastomeric element may be attached to the open channel wall via the first end and the second end may protrude into the interior space of the open channel, specifically transverse, more specifically essentially perpendicular, to a longitudinal axis of the insertion component, specifically of the insertion cannula. Before removal or retraction of the insertion component, specifically of the insertion cannula and/or the holder of the insertion cannula, from the open channel, the conductive elastomeric element may be in a compressed state and the conductive elastomeric element may be in direct contact with the surface of the insertion component, specifically of the insertion cannula and/or the holder of the insertion cannula. During and/or after removal or retraction of the insertion component, specifically of the insertion cannula and/or the holder of the insertion cannula, from the open channel, the conductive elastomeric element may be in a further compressed state or in an expanded state and the conductive elastomeric element electrically contact the at least two conductor paths, specifically the contact areas of each of the at least two conductor paths. Thereby, the conductive elastomeric element may be pressed against the contact areas of each of the at least two conductor paths. Exemplarily, the conductive elastomeric element may be made of a carbon filled elastomer such as a carbon filled silicone. However, also other materials may be possible.

Specifically, the connector element may be movably attached to a surface of the analyte sensor, specifically to a surface of the carrier of the analyte sensor. Exemplarily, the connector element may be movably attached to the surface of the analyte sensor via an adhesive. However, also other kinds of attachments may be possible. The connector element may be arranged inside the open channel. The connector element may be arranged outside of the electronics compartment. The connector element may be arranged below the at least two conductor paths, before removal or retraction of the insertion component, specifically of the insertion cannula and/or the holder of the insertion cannula. The insertion component, specifically the insertion cannula and/or the holder of the insertion cannula, may be configured for moving or displacing the connector element such that the connector element electrically contacts the at least two conductor paths, specifically the contact areas of each of the at least two conductor paths, specifically when the insertion component, specifically the insertion cannula and/or the holder of the insertion cannula, is removed or retracted from the open channel. Before retraction or removal of the insertion component, specifically of the insertion cannula and/or the holder of the insertion cannula, from the open channel, the connector element may be arranged in a distance to the at least two conductor paths. During and/or after removal or retraction of the insertion component, specifically of the insertion cannula and/or the holder of the insertion cannula, from the open channel, the connector element may electrically contact the at least two conductor paths. Specifically, during and/or after removal or retraction of the insertion component, specifically of the insertion cannula and/or the holder of the insertion cannula, from the open channel, the connector element may at least partially cover the contact areas of each of the at least two conductor paths. The insertion component, specifically the insertion cannula and/or the holder of the insertion cannula, may comprise a protrusion configured for moving the connector element in a direction of retraction of the insertion cannula such that, after insertion of the insertable portion of the analyte sensor into the body tissue, the connector element electrically contacts the at least two conductor paths. In another embodiment, during removal or retraction of the insertion component, specifically of the insertion cannula and/or the holder of the insertion cannula, from the open channel, the connector element may electrically contact the at least two conductor paths, but when the removal or retraction movement has been completed, the connector element may not electrically contact the conductor path anymore, i.e., the connector element may only transiently contact the at least two conductor paths.

The medical device may further comprise an insertion aid (or “inserter”) configured for enabling a user to drive the insertion cannula into the body tissue and to insert the insertable portion of the analyte sensor. The term “insertion aid” as used herein is a broad term and is to be given its ordinary and customary meaning to a person of ordinary skill in the art and is not to be limited to a special or customized meaning. The term specifically may refer, without limitation, to an arbitrary technical construction being configured to insert an object into another object. Therefore, the insertion aid may comprise an insertion mechanism. As further used herein, the term “mechanism” may refer to an arbitrary mechanism designed to transform input forces and movement into a desired set of output forces and movement. Specifically, the insertion mechanism may be configured such that the user may apply a force in a direction of insertion to the insertion cannula. Therefore, the insertion aid may be configured to facilitate a handling of the medical device by the user and/or to reduce application errors. The insertion aid may at least partially surround the housing, the analyte sensor, the insertion cannula, and/or the connector element. Further, the insertion aid may be at least partially coupled to the housing and/or to at least one of the removable upper cap and the removable lower cap.

The insertion aid may comprise a removable lower cover mechanically coupled to the removable lower cap. As further used herein, the term “cover” may refer to an arbitrary element that completely or at least to a large extent closes an object. Specifically, the cover may be or may comprise a shell, particularly a half-shell, surrounding the medical device. The removable lower cover may be configured such that a removal of the removable lower cover removes the removable lower cap. The insertion aid may further comprise a frame. The term “frame” may refer to an arbitrary element which may be configured to support other components of a physical construction. The frame may be displaceable on the skin of the user and may at least partially surround the housing, the analyte sensor, the insertion cannula, the removable lower cap and/or the connector element. The insertion aid may further comprise an upper cover. The upper cover may be directly or indirectly coupled to one or both of the insertion component or the removable upper cap, such that a movement of the upper cover against the frame drives the insertion cannula. The terms “lower cover” and “upper cover” may be considered as description without specifying an order and without excluding a possibility that several kinds of lower covers and upper covers may be applied.

The removable lower cover may comprise a basis which is connected to a lower part of the removable lower cap, exemplarily via a snap connection, an adhesive bonding and/or a longitudinal guide or transferring force. The basis may comprise gripping surfaces for removing the removable lower cap. The basis may at the same time be a cover for the adhesive surface. This may lead to an extended shelf-life of the adhesive surface. By removing of the removable lower cover the removable lower cap may be opened, the insertion component and the analyte sensor may be exposed and the adhesive surface may be exposed at the same time.

The medical device may further comprise a retraction mechanism for retracting the insertion cannula after insertion of the insertable portion of the analyte sensor into the body tissue. The term “retraction mechanism” may generally refer to an arbitrary construction which is configured to move an object in an opposite direction of a direction in which the object may have been moved before the retraction mechanism is applied. Therefore, the retraction mechanism may comprise a retraction contact spring element The retraction contact spring element may be biased in order to retract the insertion cannula from the body tissue. The retraction mechanism may at least partially be comprised within the removable upper cap and/or within the upper cover.

In a further aspect, a medical device system is disclosed. The medical device system comprises the medical device as described above or as will further be described below in more detail.

Further, the medical device system comprises a sensor controller which is coupled to the analyte sensor. The sensor controller is configured to receive analyte sensor data from the analyte sensor. The sensor controller may specifically comprise at least one data processing unit, such as a processor. Further, the sensor controller may comprise at least one volatile or non-volatile data storage. The sensor controller may comprise at least one interface configured for entering commands and/or for outputting information. The at least one interface may comprise a wired interface and/or a wireless interface for unidirectionally or bidirectionally exchanging data or commands, specifically between the sensor controller and at least one further device.

Further, the medical device system comprises a remote control which is configured to receive sensor data from the sensor controller and to process and/or display sensor data. The sensor controller may be configured to communicate the analyte sensor data to the remote control. The term “communication” as used herein is a broad term and is to be given its ordinary and customary meaning to a person of ordinary skill in the art and is not to be limited to a special or customized meaning. The term specifically may refer, without limitation, to a process of transferring information. In particular, information from a computational device may be transferred such as by sending or outputting information, e.g., onto another device. Specifically, a communication interface may be provided. The communication interface may specifically provide means for transferring or exchanging information. In particular, the communication interface may provide a data transfer connection, e.g., Bluetooth, NFC, inductive coupling or the like.

Specifically, the medical device system may comprise a user interface. The term “user interface” as used herein is a broad term and is to be given its ordinary and customary meaning to a person of ordinary skill in the art and is not to be limited to a special or customized meaning. The term may refer, without limitation, to an element or device which is configured for interacting with its environment, such as for the purpose of unidirectionally or bidirectionally exchanging information, such as for exchange of one or more of data or commands. For example, the user interface may be configured to share information with a user and to receive information by the user. The user interface may be a feature to interact visually with a user, such as a display, or a feature to interact acoustically with the user. The user interface, as an example, may comprise one or more of: a graphical user interface; a data interface, such as a wireless and/or a wire-bound data interface.

In a further aspect, a further medical device is disclosed. The further medical device comprises an analyte sensor having an insertable portion adapted for at least partially being inserted into a body tissue of a user. The analyte sensor is configured for detecting the analyte in the body fluid. Further, the further medical device comprises an insertion component comprising an insertion cannula. The analyte sensor is at least partially placed inside the insertion cannula. Further, the further medical device comprises an electronics unit comprising a sensor circuit. Further, the further medical device comprises an housing having an electronics compartment with the electronics unit at least partially received therein. The housing further comprises an open channel which at least partially surrounds the analyte sensor and the insertion component. With regard to the analyte sensor, the insertion component, the electronics unit and the housing, reference is made to the description above.

Further, the further medical device comprises a pushbutton switch. The insertion component is configured for being retracted from the open channel thereby triggering an opening of the pushbutton switch such that the sensor circuit is activated.

The term “pushbutton switch” as used herein is a broad term and is to be given its ordinary and customary meaning to a person of ordinary skill in the art and is not to be limited to a special or customized meaning. The term specifically may refer, without limitation, to an arbitrary operating element which is configured for closing and interrupting an electrical circuit by means of a push-button. This controls whether a current flows in the electrical circuit or not. Specifically, the push-button switch may be deactivated by pressing a movable element such as a button and activated by releasing it. When released, the pushbutton switch may establish an electrical connection. When pressed, the pushbutton switch may interrupt the electrical connection. In order for the pushbutton switch to function, a spring may be applied which takes up a released position after the movable element is pressed. For the pushbutton switch, the released position which may also be referred to as an open state, may be a normal position or state. The electrical circuit may be interrupted only by pressing the movable element such as the button. The electrical circuit may remain interrupted only as long as the movable element remains pressed. If the movable element is released, the electrical circuit is closed.

The push-button switch may comprise a pushbutton switch housing and a movable element. The movable element may be configured for being movable transverse, specifically perpendicular, to the direction of insertion of the analyte sensor and/or to a longitudinal axis of the insertion component, specifically of the insertion cannula. The movable element may protrude into the interior space enclosed by the open channel. The pushbutton switch may be arranged within the open channel. Specifically, the pushbutton switch may be attached to the open channel wall, specifically fixedly, specifically to the open channel wall facing the interior space of the open channel.

Before insertion of the insertion cannula, e.g., before retraction or removal of the insertion component, specifically of the insertion cannula and/or the holder of the insertion cannula, from the open channel, the insertion component, specifically the insertion cannula and/or the holder of the insertion cannula, may compress the movable element into the pushbutton switch housing thereby interrupting the sensor circuit. By retracting or removing the insertion component, specifically the insertion cannula and/or the holder of the insertion cannula, from the open channel the movable element may be released. Specifically, the movable element may move out of the pushbutton switch housing which causes the sensor circuit to close. In turn, the electronics unit may be switched on as power from the battery is received.

In a further aspect of this disclosure, a method of using the medical device according to any embodiment as described above or as further described below is disclosed. The methods comprise the method steps as given in the independent claims and as listed as follows. The method steps may be performed in the given order. However, other orders of the method steps are feasible. Further, one or more of the method steps may be performed in parallel and/or on a timely overlapping fashion. Further, one or more of the method steps may be performed repeatedly. Further, additional method steps may be present which are not listed.

The method comprises the following steps:
a) providing the medical device;
b) inserting the analyte sensor into the body tissue; and
c) removing the insertion cannula from the open channel, whereby an electrically contacting of the at least two conductor paths of the analyte sensor by the connector element is triggered such that the sensor circuit is activated.

The proposed medical device and the method of using a medical device provide many advantages over known devices and methods.

The medical device, specifically a wearable, specifically an integrated, analyte sensor system may be disclosed comprising the analyte sensor having the insertable portion adapted for at least partially being inserted into the body tissue of the user, the analyte sensor being configured to detect the analyte in the body fluid of a patient. The analyte sensor may comprise the carrier, specifically a carrier material, the working electrode configured for detecting the analyte, the counter electrode and/or the reference electrode and the at least two conductor paths which, when connected to each other by an element such as a mechanical switch element, may be configured to establish an electrical contact between the battery and the electronic component of the electronics unit. The medical device further comprises the insertion cannula, wherein the analyte sensor at least partially is placed inside the insertion cannula. The medical device further comprises the housing which may comprise the upper side and the lower side and the open channel which may connect the upper side and the lower side. The open channel may be sealed off from the electronics compartment which may be formed by the upper side and the lower side and by the open channel wall of the open channel. The electronics compartment may comprise the electronics unit which may be configured to connect with the analyte sensor. Further, the medical device may comprise a sterility capsule encompassing the insertion cannula and a part of the analyte sensor, wherein the sterility capsule may be formed by the removable upper cap which comprises the insertion cannula, by the wall of the open channel and by the removable lower cap. The removable lower cap may be configured for removal before insertion. The insertion cannula may be attached to the removable upper cap. The removable upper cap may be configured for removal after insertion, thereby removing the insertion cannula. The insertion cannula and/or the removable upper cap may be configured to move the connector element which may be configured to electrically connect the at least two conductor paths.

The sensor circuit may be activated by the insertion cannula. The retraction of the insertion cannula may trigger the connector element to electrically connect the at least two conductor paths. Specifically, sensor circuit may be activated by insertion cannula retraction via triggering a connector element to electrically connect at least two conductor paths.

Advantageously, the at least two conductor paths may be part of the analyte sensor itself. Thus, a simple construction setup may be achieved. Further, specifically by using a simple switch mechanism that may be triggered by the retraction movement of the insertion cannula and/or the removable upper cap as well as by the at least two conductor paths of the analyte sensor, it may be possible to keep the electronic components of the electronics unit, specifically sensor electronics, in an off-state during an entire storage life where no power is consumed until the analyte sensor is eventually inserted. At the same time, the switch mechanism may not require to introduce additional openings that penetrate the electronics compartment for the analyte sensor, beyond the analyte sensor itself.

This way, a size of the battery on one hand and of the medical device as a whole may be significantly reduced compared to hitherto known devices where the electronics unit switches back and forth between a low power sleep mode and a high power test mode. Moreover, there may be a reduced risk of an incomplete sealing of the electronics compartment and the sterility capsule. Finally, there may be a reduction of costs for manufacturing.

Summarizing and without excluding further possible embodiments, the following embodiments may be envisaged:
Embodiment 1: A medical device for detecting an analyte in a body fluid, the medical device comprising:
   an analyte sensor having an insertable portion adapted for at least partially being inserted into a body tissue of a user, wherein the analyte sensor is configured for detecting the analyte in the body fluid, wherein the analyte sensor comprises at least two conductor paths configured for activating a sensor circuit, wherein the analyte sensor further comprises at least two electrodes each comprising an electrode conductor path configured for transmitting a sensor current for detecting the analyte;
   an insertion component comprising an insertion cannula, wherein the analyte sensor is at least partially placed inside the insertion cannula;
   an electronics unit comprising the sensor circuit;
   an housing having an electronics compartment with the electronics unit at least partially received therein, wherein the housing further comprises an open channel which at least partially surrounds the analyte sensor and the insertion component; and
   a connector element, wherein the insertion component is configured for being retracted from the open channel thereby triggering an electrically contacting of the conductor paths of the analyte sensor by the connector element such that the sensor circuit is activated.
Embodiment 2: The medical device according to the preceding claim, wherein the sensor circuit comprises an electrical energy reservoir, specifically a battery, wherein the connector element is configured for establishing an electrical connection between the electrical energy reservoir and electronic components of the sensor circuit by electrically contacting the at least two conductor paths of the analyte sensor.
Embodiment 3: The medical device according to any one of the preceding claims, wherein the electronics unit comprises a printed circuit board with the sensor circuit disposed thereon.
Embodiment 4: The medical device according to any one of the preceding claims, wherein, before insertion of the insertable portion of the analyte sensor into the body tissue, the sensor circuit is in an inactive state and wherein, after insertion of the insertable portion of the analyte sensor into the body tissue, the sensor circuit is in an active state.
Embodiment 5: The medical device according to any one of the preceding claims, wherein, before retraction or removal of the insertion cannula from the open channel, the sensor circuit is in an inactive state and wherein, during and/or after retraction or removal of the insertion cannula from the open channel, the sensor circuit is in an active state.
Embodiment 6: The medical device according to any one of the preceding claims, wherein the connector element is configured for electrically contacting the at least two conductor paths of the analyte sensor by a movement, a displacement, a relaxation or an expansion of the connector element or a component of the connector element.
Embodiment 7: The medical device according to any one of the preceding claims, wherein the housing comprises an upper side and a lower side, wherein the open channel connects the upper side and the lower side.
Embodiment 8: The medical device according to the preceding claim, wherein the insertable portion of the analyte sensor extends downwardly in the open channel beyond the lower side of the housing.
Embodiment 9: The medical device according to any one of the preceding claims, wherein the at least two conductor paths are arranged parallel to each other.
Embodiment 10: The medical device according to any one of the preceding claims, wherein the at least two conductor paths are arranged in a distance to each other.
Embodiment 11: The medical device according to any one of the preceding claims, wherein each of the at least two conductor paths comprises a contact area, wherein the connector element is configured for electrically contacting the contact areas of each of the at least two conductor paths.
Embodiment 12: The medical device according to the preceding claim, wherein the contact areas are arranged opposite to each other.
Embodiment 13: The medical device according to any one of the two preceding claims, wherein the contact areas are exposed by laser abrasion.
Embodiment 14: The medical device according to any one of the three preceding claims, wherein the contact areas are located on or disposed on a surface of the analyte sensor.
Embodiment 15: The medical device according to the preceding claim, wherein the contact areas are elevated relative to a surrounding surface of the analyte sensor.
Embodiment 16: The medical device according to any one of the five preceding claims, wherein the contact areas respectively comprise a soldered area.
Embodiment 17: The medical device according to any one of the six preceding claims, wherein the contact areas of each of the at least two conductor paths are arranged opposite to the connector element.
Embodiment 18: The medical device according to any one of the seven preceding claims, wherein the contact areas of each of the at least two conductor paths are arranged opposite to contact areas of the connector element.
Embodiment 19: The medical device according to any one of the eight preceding claims, wherein the contact areas of each of the at least two conductor paths are arranged outside of the electronics compartment.
Embodiment 20: The medical device according to any one of the preceding claims, wherein the connector element is selected from the group consisting of: a contact spring element, specifically a bent contact spring element, specifically a leaf contact spring element; a conductive elastomeric element.
Embodiment 21: The medical device according to the preceding claim, wherein, before retraction or removal of the insertion cannula from the open channel, the connector element is in direct contact with a surface of the insertion component, wherein, during and/ or after retraction or removal of the insertion cannula from the open channel, the connector element electrically contacts the at least two conductor paths.
Embodiment 22: The medical device according to any one of the two preceding claims, wherein the connector element is the contact spring element, wherein, before retraction or removal of the insertion cannula from the open channel, the contact spring element is in a tensioned state and the contact spring element is in direct contact with the surface of the insertion cannula, wherein during and/or after removal of the insertion cannula from the open channel, the contact spring element is in a further tensioned state or in a relaxed state and the contact spring element electrically contacts the at least two conductor paths.
Embodiment 23: The medical device according to any one of the three preceding claims, wherein the connector element is the conductive elastomeric element, wherein, before retraction or removal of the insertion cannula from the open channel, the conductive elastomeric element is in a compressed state and the conductive elastomeric element is in direct contact with the surface of the insertion cannula, wherein, during and/or after retraction or removal of the insertion cannula from the open channel, the conductive elastomeric element is in a further compressed state or in an expanded state and the conductive elastomeric element electrically contacts the at least two conductor paths.
Embodiment 24: The medical device according to any one of the preceding claims, wherein the connector element is movably attached to a surface of the analyte sensor.
Embodiment 25: The medical device according to the preceding claim, the insertion cannula is configured for moving the connector element such that the connector element electrically contacts the at least two conductor paths.
Embodiment 26: The medical device according to any one of the two preceding claims, wherein, before retraction or removal of the insertion cannula from the open channel, the connector element is arranged in a distance to the at least two conductor paths, wherein, during and/or after retraction or removal of the insertion cannula from the open channel, the connector element electrically contacts the at least two conductor paths.
Embodiment 27: The medical device according to any one of the three preceding claims, wherein the insertion cannula comprises a protrusion configured for moving the connector element in a direction of retraction of the insertion cannula such that, after insertion of the insertable portion of the analyte sensor into the body tissue, the connector element electrically contacts the at least two conductor paths.
Embodiment 28: The medical device according to any one of the four preceding claims, wherein the connector element is movably attached to the surface of the analyte sensor via an adhesive.
Embodiment 29: The medical device according to any one of the five preceding claims, wherein the connector element is arranged below the at least two conductor paths.
Embodiment 30: The medical device according to any one of the preceding claims, wherein the open channel is formed by an open channel wall.
Embodiment 31: The medical device according to the preceding claim, wherein the electronics compartment is at least partially formed by the open channel wall.
Embodiment 32: The medical device according to any one of the two preceding claims, wherein the open channel wall comprises an opening, wherein the analyte sensor passes through the opening.
Embodiment 33: The medical device according to the preceding claim, wherein the opening is a sealed opening.
Embodiment 34: The medical device according to any one of the four preceding claims, wherein the connector element is attached to a surface of the open channel wall, wherein, specifically, the connector element is fixedly attached to the surface of the open channel wall.
Embodiment 35: The medical device according to the preceding claim, wherein the connector element is attached to the surface of the open channel wall facing an interior space enclosed by the open channel.
Embodiment 36: The medical device according to any one of two preceding claims, wherein, before retraction or removal of the insertion cannula from the open channel, the insertion cannula is configured as a non-conductive spacer between the connector element and the at least two conductor paths.
Embodiment 37: The medical device according to any one of three preceding claims, wherein, before retraction or removal of the insertion cannula from the open channel, the connector element is compressed by or tensioned by an outer surface of the insertion cannula.
Embodiment 38: The medical device according to any one of the preceding claims, wherein the analyte sensor is partially received in the electronics compartment and partially located outside of the electronics compartment, wherein the insertable portion of the analyte sensor is located outside of the electronics compartment.
Embodiment 39: The medical device according to any one of the preceding claims, wherein the medical device further comprises a removable upper cap, wherein the insertion cannula is attached to the removable upper cap, specifically wherein the insertion cannula is fixedly attached to the removable upper cap.
Embodiment 40: The medical device according to the preceding claim, wherein the removable upper cap is configured for removal after insertion of the insertable portion of the analyte sensor into the body tissue.
Embodiment 41: The medical device according to the preceding claim, wherein the removable upper cap at least partially surrounds the insertion cannula.
Embodiment 42: The medical device according to any one of the three preceding claims, wherein the removable upper cap is removably connected to the housing via a connection, specifically via at least one of a screwing connection, a bayonet connection.
Embodiment 43: The medical device according to any one of the preceding claims, wherein the medical device further comprises a removable lower cap, wherein the removable lower cap is configured for removal before insertion of the insertable portion of the analyte sensor into the body tissue.
Embodiment 44: The medical device according the preceding claim, wherein the removable lower cap is a sterile cap configured to provide sterile packaging for the insertable portion of the analyte sensor, such that the insertable portion is sealed against a surrounding environment.
Embodiment 45: The medical device according to any one of the preceding claims, wherein the open channel at least partially circumferentially surrounds the analyte sensor and the insertion cannula.
Embodiment 46: The medical device according to any one of the preceding claims, wherein the medical device further comprises a retraction mechanism for retracting the insertion cannula after insertion of the insertable portion of the analyte sensor into the body tissue.
Embodiment 47: The medical device according to any one of the two preceding claims, wherein the retraction mechanism comprises a retraction contact spring element, more preferably a retraction contact spring element disposed in between the housing and the insertion cannula and biased in order to retract the insertion cannula from the body tissue.
Embodiment 48: The medical device according to any one of the preceding claims, wherein the analyte sensor comprises a carrier, specifically a substrate, wherein the at least two conductor paths are disposed on the carrier.
Embodiment 49: The medical device according to any one of the preceding claims, wherein the at least two electrodes are a working electrode configured for detecting the analyte and a further electrode, wherein the further electrode is selected from the group consisting of: a counter electrode, a reference electrode.
Embodiment 50: The medical device according to any one of the preceding claims, wherein the medical device comprises an adhesive surface for attachment to a user's skin.
Embodiment 51: The medical device according to any one of the preceding claim, wherein the adhesive surface is directly or indirectly attached to the housing.
Embodiment 52: The medical device according to any one of the two preceding claims, wherein the adhesive surface has a shape of a circular ring surrounding the analyte sensor.
Embodiment 53: The medical device according to any one of the three preceding claims, wherein the adhesive surface comprises at least one of a plaster or an adhesive strip.
Embodiment 54: The medical device according to any one of the preceding claims, wherein the medical device is a disposable medical device.
Embodiment 55: The medical device according to any one of the preceding claims, wherein the electronics unit is a single-use electronics unit.
Embodiment 56: The medical device according to any one of the preceding claims, wherein the analyte sensor, the insertion cannula, the electronics unit, the housing and the connector element form a pre-assembled single unit.
Embodiment 57: The medical device according to any one of the preceding claims, wherein the housing comprises at least two housing portions, specifically a lower housing portion and an upper housing portion.
Embodiment 58: The medical device according to the preceding claim, wherein the upper housing portion and the lower housing portion are connected via one or more of a form-fit connection, a force-fit connection or a connection by material engagement, more specifically by a connection using an adhesive and/or a bonding.
Embodiment 59: The medical device according to any one of the two preceding claims, wherein the upper housing portion and the lower housing portion form an encapsulation for electronic components of the electronics unit.
Embodiment 60: A method of using the medical device according to any one of the preceding claims, the method comprising:
   a) providing the medical device;
   b) inserting the analyte sensor into the body tissue; and
   c) removing the insertion cannula from the open channel, whereby an electrically contacting of the at least two conductor paths of the analyte sensor by the connector element is triggered such that the sensor circuit is activated.
Embodiment 61: A medical device system, comprising:
   a medical device according to any one of the preceding embodiments referring to a medical device;
   a sensor controller which is coupled to the analyte sensor, wherein the sensor controller is configured to receive analyte sensor data from the analyte sensor; and
   a remote control which is configured to receive sensor data from the sensor controller and to process and/or display sensor data.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIGS. 1A to 1C show an exemplary embodiment of an analyte sensor according to this disclosure in a top view (FIG. 1A) as well as in cross-sectional views of an analyte sensor (FIGS. 1B and 1C);

FIGS. 2A and 2B show an exemplary embodiment of a medical device according to this disclosure after insertion of the analyte sensor (FIG. 2A) and after insertion of the analyte sensor and during removal of the insertion cannula (FIG. 2B);

FIGS. 3A and 3B show a further exemplary embodiment of a medical device according to this disclosure after insertion of the analyte sensor (FIG. 3A) and after insertion of the analyte sensor and during removal of the insertion cannula (FIG. 3B);

FIGS. 4A to 4C show an exemplary embodiment of a medical device according to this disclosure after insertion of the analyte sensor (FIG. 4A), during removal of the insertion cannula (FIG. 4B) and after removal of the insertion cannula (FIG. 4C);

FIGS. 5A and 5B show a further exemplary embodiment of a medical device after insertion of the analyte sensor (FIG. 5A) and after insertion of the analyte sensor and during removal of the insertion cannula (FIG. 5B);

FIG. 6 shows a schematic embodiment of a medical device system according to this disclosure.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

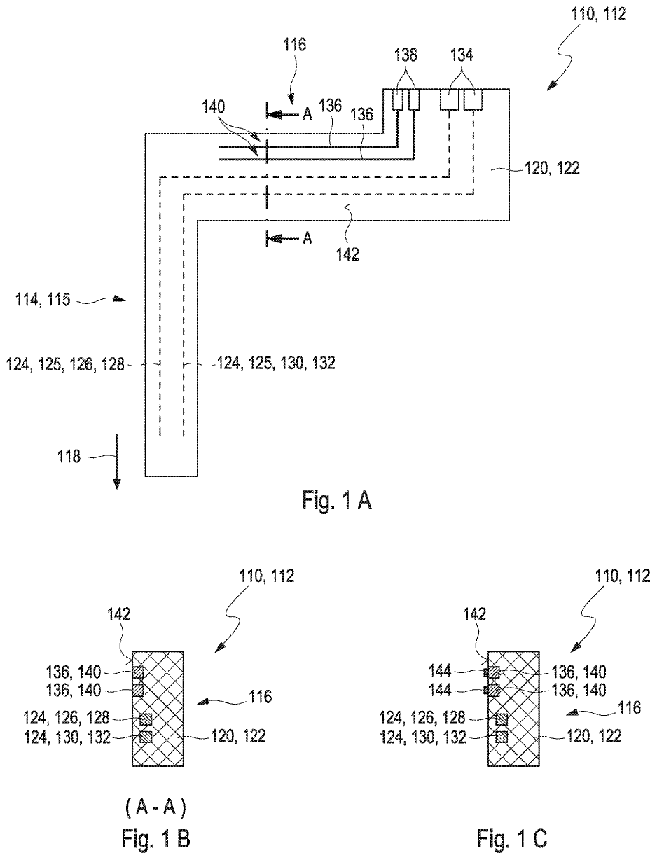

FIG. 1A shows an exemplary embodiment of an analyte sensor 110 according to this disclosure in a top view. The analyte sensor 110 may be an electrochemical sensor 112. The analyte sensor 110 may have an elongate shape. Specifically, the analyte sensor 110 may be straight, curved or bent. Specifically, the analyte sensor 110 may have an in vivo proximal portion 114 and an ex vivo distal portion 116. The in vivo proximal portion 114 may be referred to as insertable portion 115. The in vivo proximal portion 114 may be configured for being inserted into the body tissue of the user. The ex vivo distal portion 116 may be configured for staying outside of the body tissue of the user. The ex vivo distal portion 116 and the in vivo proximal portion 114 may be arranged transverse, specifically essentially perpendicular, to each other. The in vivo proximal portion 114 may extend along a direction of insertion as indicated with arrow 118. The direction of insertion may be transverse, specifically essentially perpendicular, to a skin site of the user.

The analyte sensor 110 may comprise a carrier 120 which may specifically be or may comprise a substrate 122. In the embodiment according to FIG. 1A, the analyte sensor 110 comprises two electrodes 124 each comprising an electrode conductor path 125. A first electrode 126 may be a working electrode 128. A second electrode 130 may be a counter or reference electrode 132. The electrodes 124 may be attached to the carrier 120 or may be disposed within the carrier 120. The electrodes 124 may extend along the carrier 120. Specifically, the electrodes 124 may respectively be formed as a line. The electrodes 124 may be arranged parallel and at a distance from each other. The first electrode 126 and the second electrode 130 may respectively comprise a contact pad 134 for electrically connecting with one or more electronics components of an electronics unit.

Further, the analyte sensor 110 may comprise at least two conductor paths 136. The at least two conductor paths 136 may be attached to the carrier 120 or may be disposed on the carrier 120. The at least two conductor paths 136 may specifically be arranged on the ex vivo distal portion 116 of the analyte sensor 110. After insertion of the analyte sensor 110, specifically of the in vivo proximal portion 114 of the analyte sensor 110, the at least two conductor paths 136 may stay outside of the body tissue. The at least two conductor paths 136 may respectively be formed as a line. The at least two conductor paths 136 may be arranged parallel and at a distance from each other so that no contact is unintentionally established. Each of the at least two conductor paths 136 may comprise a contact pad 138 for connecting with one or more electronics components of an electronics unit or with the energy source such as a battery. Further, the at least two conductor paths 136 may respectively comprise a contact area 140. Further details on the contact areas 140 are provided in FIGS. 1B and 1C.

FIGS. 1B and 1C respectively show cross-sectional views of the analyte sensor 110 according to FIG. 1A (section A-A, see FIG. 1A). FIGS. 1B and 1C respectively show different embodiments. As can be seen in FIGS. 1B and 1C, the contact areas 140 of the at least two conductor paths 136 may be at least partially located superficially on and/or near a surface 142 of the analyte sensor 110, specifically of the carrier 120. As illustrated in FIG. 1B, the contact areas 140 may exemplarily be exposed by laser abrasion. To increase access of the at least two conductor paths 136 for contacting by a connector element, the contact areas 140 may be elevated relative to the surface 142 such as by adding a soldered contact element 144 to the exposed contact areas 140 of the at least two conductor paths 136, such as illustrated in FIG. 1C. It is also conceivable that an even more defined contacting surface can be provided by mounting and/or soldering a contact element 144 onto the contract areas 140.

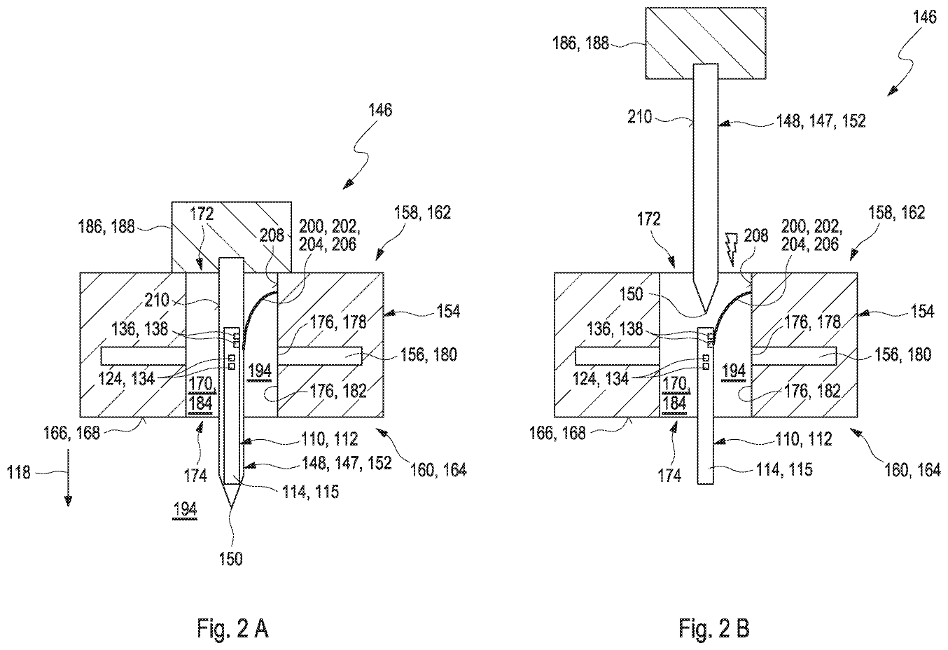

FIGS. 2A and 2B show an exemplary embodiment of a medical device 146 according to this disclosure after insertion of the analyte sensor 110 and the insertion cannula 148 (FIG. 2A) and after insertion of the analyte sensor 110 and during removal of the insertion cannula 148 (FIG. 2B). The medical device 146 comprises the analyte sensor 110 as illustrated in FIGS. 1A to 1C. Thus, reference is made to the description of FIGS. 1A to 1C above.

The medical device 146 further comprises an insertion component 147 comprising an insertion cannula 148. The analyte sensor 110 is at least partially placed inside the insertion cannula 148. The insertion cannula 148 may comprise a tip 150 for inserting the analyte sensor 110 at least partially into the body tissue. The insertion cannula 148 may specifically be a slotted needle 152. The insertion cannula 140 may be configured to be inserted vertically relative to the body tissue of the user.

The medical device 146 further comprises a housing 154 having an electronics compartment 156 with an electronics unit (not illustrated in FIGS. 2A and 2B) at least partially received therein. The housing 154 may comprise an upper side 158 and a lower side 160. Specifically, the upper side 158 may refer to a distal side 162 of the housing 154. Specifically, the lower side 160 may refer to a proximal side 164 of the housing 154. The lower side 160 may comprise a surface 166 for being placed on a user's skin. Specifically, the medical device 146 may comprise an adhesive surface 168 for attachment to the user's skin.

The housing 154 further comprises an open channel 170 which at least partially surrounds the analyte sensor 110 and the insertion cannula 148. The open channel 170 may specifically be an essentially straight channel. The open channel 170 may extend along the direction of insertion of the analyte sensor 110, such as indicated with arrow 118. The open channel 170 may specifically have an upper side opening 172 and an opposing lower side opening 174. The upper side opening 172 may be located on the upper side 158 of the housing 154 facing away from the body tissue and the lower side opening 174 may be located on the lower side 160 of the housing 154 facing the body tissue. The open channel 170 may connect the upper side 158 and the lower side 160. The insertable portion 115 of the analyte sensor 110 may extend downwardly in the open channel beyond the lower side 160 of the housing 154.

The open channel 170 may be formed by an open channel wall 176. The electronics compartment 156 may be at least partially formed by the open channel wall 176. Thus, the open channel wall 176 may have a first side 178 facing an interior space 180 of the electronics compartment 156 and an opposing second side 182 facing an interior space 184 of the open channel 170.

The open channel wall 176 may comprise an opening (not shown in FIGS. 2A to 2B), wherein the analyte sensor 110 passes through the opening which can be a sealed opening. The analyte sensor 110 may be partially received inside the electronics compartment 156 while another part of the analyte sensor 110 may be partially received outside the electronics compartment 156, such as within the open channel 170. The insertable portion 115 of the analyte sensor 110 may be located outside of the electronics compartment 156. The opening may specifically be a sealed opening.

The medical device 146 may comprise a removable upper cap 186. The removable upper cap 186 may be configured for removal after insertion of the insertable portion 115 of the analyte sensor 110 into the body tissue. The removable upper cap 186 may specifically be or may comprise an holder 188 for the insertion cannula 148. The insertion cannula 148 may be connected to the holder 188, specifically fixedly.

Further, the medical device 146 may comprise a removable lower cap (not shown). The removable lower cap may be configured for removal before insertion of the insertable portion 115 of the analyte sensor 110 into the body tissue. The removable lower cap may specifically be a sterility cap. The sterility cap may be configured to provide sterile packaging for the insertable portion 115 of the analyte sensor 110, such that the insertable portion 115 is sealed against a surrounding environment.

The removable lower cap and the removable upper cap 186 may be arranged or located on opposing sides of the housing 154, specifically of the open channel 170. Specifically, the removable lower cap may be arranged on the lower side of 160 the housing 154. The removable lower cap may be removably connected, specifically attached, to the lower side 160 of the housing 154. The removable upper cap 186 may be arranged on the upper side 158 of the housing 154. The removable upper cap 186 may be removably connected, specifically attached, to the upper side 158 of the housing 154.

The removable upper cap 186 and the removable lower cap may be reversibly or irreversible connected to the housing 154. The removable lower cap, the removable upper cap 186 and the open channel 170 may form a sensor compartment 194 for at least partially receiving the analyte sensor 110. The analyte sensor 110 may be partially received in the electronics compartment 156 and partially received in the sensor compartment 194. The sensor compartment 194 may be a sealed compartment, specifically a sterile compartment, before the removable lower cap is removed from the housing 154.

The electronics unit may comprise a circuit carrier, preferably a printed circuit board. Similarly, the optional energy reservoir, such as a battery, may directly or indirectly be attached to the printed circuit board. A sensor circuit may be disposed on the printed circuit board. These features are not illustrated in FIGS. 2A and 2B.

The medical device 146 further comprises a connector element (also referred to herein as a “connector”) 200 arranged inside the open channel 170 of the housing 154. The connector element 200 may be configured for electrically contacting the at least two conductor paths 136 of the analyte sensor 110.

In the embodiment according to FIGS. 2A and 2B, the connector element 200 is a contact spring element 202, specifically a bent contact spring element 204, more specifically a leaf contact spring element 206. The contact spring element 202 may be fixed attached to a surface 208 of the open channel wall 176.

Before retraction or removal of the insertion cannula 148 from the open channel 170, such as illustrated in FIG. 2A, the contact spring element 202 may be in direct contact with a surface 210 of the insertion cannula 148. Thereby, the contact spring element 202 may be in a tensioned state and the contact spring element 202 may be arranged in a distance to the at least two conductor paths 136 of the analyte sensor 110. The sensor circuit may be in an inactive state.

During retraction or removal of the insertion cannula 148 from the open channel 170, such as illustrated in FIG. 2B, the contact spring element 202 electrically contacts the at least two conductor paths 136. The contact spring element 202 may be in a further tensioned state or in a relaxed state and the contact spring element 202 may electrically contact the at least two conductor paths 136. Thus, the sensor circuit may be closed and may be in an active state.

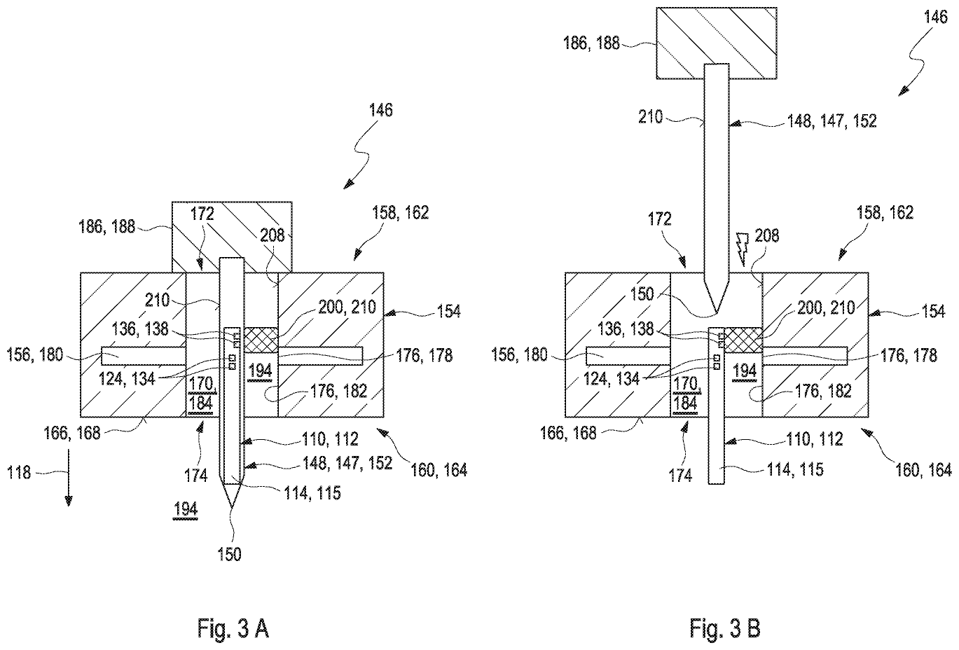

FIGS. 3A and 3B show an exemplary embodiment of a further medical device 146 according to this disclosure after insertion of the analyte sensor 110 (FIG. 3A) and after insertion of the analyte sensor 110 and during removal of the insertion cannula 148 (FIG. 3B). The medical device 146 comprises the analyte sensor 110 as illustrated in FIGS. 1A to 1C. Thus, reference is made to the description of FIGS. 1A to 1C above. Further, the medical device 146 according to FIGS. 3A and 3B corresponds at least partially to the medical device 146 according to FIGS. 2A and 2B. Thus, reference is made to the description of FIGS. 2A and 2B above.

In the embodiment according to FIGS. 3A and 3B, the connector element 200 is a conductive elastomeric element 210. The conductive elastomeric element 210 may be fixed attached to the surface 208 of the open channel wall 176.

Before retraction or removal of the insertion cannula 148 from the open channel 170, such as illustrated in FIG. 3A, the conductive elastomeric element 210 may be in direct contact with the surface 210 of the insertion cannula 148. Thereby, the conductive elastomeric element 210 may be in a compressed state and the conductive elastomeric element 210 may be arranged in a distance to the at least two conductor paths 136 of the analyte sensor 110. The sensor circuit may be in an inactive state.

During retraction or removal of the insertion cannula 148 from the open channel 170, such as illustrated in FIG. 3B, the conductive elastomeric element 210 electrically contacts the at least two conductor paths 136. The conductive elastomeric element 210 may be in a further compressed state or in a relaxed state and the conductive elastomeric element 210 may electrically contact the at least two conductor paths 136. Thus, the sensor circuit may be closed and may be in an active state.

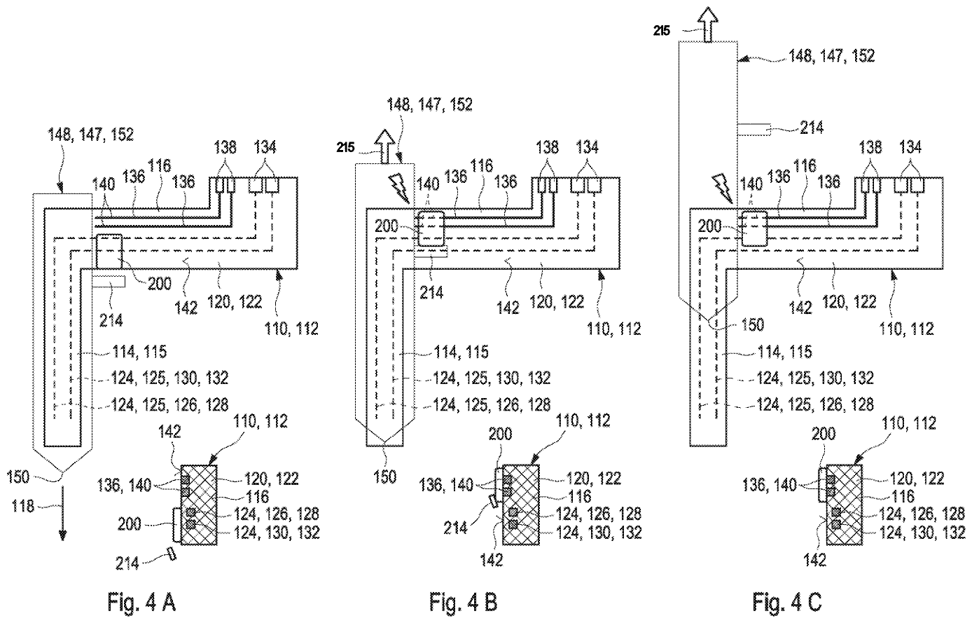

FIGS. 4A to 4C show an exemplary embodiment of a further medical device 146 according to this disclosure after insertion of the analyte sensor 110 and the insertion cannula 148 (FIG. 4A) and after insertion of the analyte sensor 110, during removal of the insertion cannula 148 and after removal of the insertion cannula 148 (FIG. 4C). In the FIGS. 4A to 4C, the medical device 146 is respectively shown in a top view (above) and in a cross-sectional view (below). The medical device 146 comprises the analyte sensor 110 as illustrated in FIGS. 1A to 1C. Thus, reference is made to the description of FIGS. 1A to 1C above. Further, the medical device 146 according to FIGS. 4A to 4C corresponds at least partially to the medical device 146 according to FIGS. 2A and 2B. Thus, reference is made to the description of FIGS. 2A and 2B above.

In the embodiment according to FIGS. 4A to 4C, the connector element 200 is movably attached to a surface 212 of the analyte sensor 110 such as via an adhesive. The insertion cannula 148 may be configured for moving the connector element 200 such that the connector element 200 electrically contacts the at least two conductor paths 136 of the analyte sensor 110. For this purpose, the insertion cannula 148 may comprise a protrusion 214. The protrusion 214 may be configured for moving the connector element 200 in a direction of retraction of the insertion cannula 148, such as indicated with arrow 215.

Before retraction or removal of the insertion cannula 148 from the open channel 170, such as illustrated in FIG. 4A, the connector element 200 may be arranged in a distance to the at least two conductor paths 136. The connector element 200 may be arranged below the at least two conductor paths 136. Further, the connector element 200 may be arranged above the protrusion 214.

During and after retraction or removal of the insertion cannula 148 from the open channel 170, such as illustrated in FIGS. 4B and 4C, the connector element 200 electrically contacts the at least two conductor paths 136. Specifically, the connector element 200 may be disposed at least partially on both of the at least two conductor paths 136.

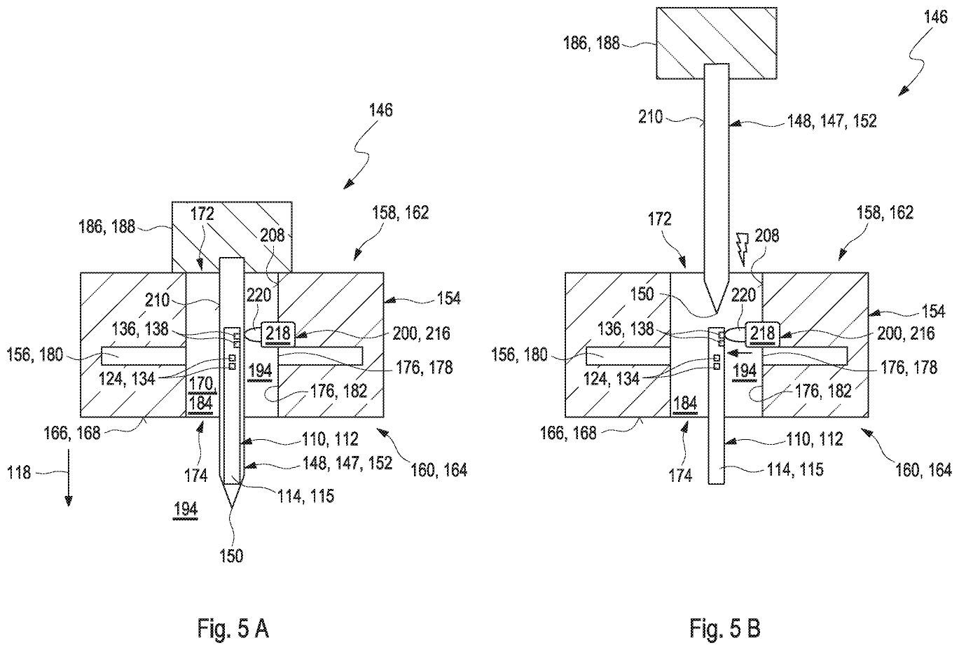

FIGS. 5A and 5B show an exemplary embodiment of a further medical device 146 after insertion of the analyte sensor 110 and of the insertion cannula 148 (FIG. 5A) and after insertion of the analyte sensor 110 and during removal of the insertion cannula 148 (FIG. 5B). In the FIGS. 5A and 5B, the medical device 146 is respectively shown in a cross-sectional view.

The medical device 146 may comprise the analyte sensor 110 which corresponds at least partially to the analyte sensor 110 as illustrated in FIGS. 1A to 1C. Thus, reference is made to the description of FIGS. 1A to 1C above. In contrast to the analyte sensor 110 as illustrated in FIGS. 1A to 1C, the analyte sensor 110 as illustrated in FIGS. 5A and 5B does not comprise the at least two conductor paths 136.

Further, the medical device 146 according to FIGS. 5A and 5B corresponds at least partially to the medical device 146 according to FIGS. 2A and 2B. Thus, reference is made to the description of FIGS. 2A and 2B above.

In the embodiment according to FIGS. 5A and 5B, the connector element 200 may be a pushbutton switch 216. The pushbutton switch 216 may be arranged within the open channel 170. Specifically, the pushbutton switch 216 may be fixed attached to the open channel wall 176. The pushbutton switch 216 may comprise a pushbutton switch housing 218 and a movable element 220. The movable element 220 may be configured for being movable transverse, specifically perpendicular, to the direction of insertion of the analyte sensor 110, such as indicated with arrow 118. Before insertion of the insertion cannula 148, e.g., before retraction or removal of the insertion cannula 148 from the open channel 170, such as illustrated in FIG. 5A, the insertion cannula 148 may compress the movable element 220 into the pushbutton switch housing 218, thereby interrupting the sensor circuit. This state may also refer to an ex-factory state. By retracting the insertion cannula 148 from the open channel 170, the movable element 220 may move out of the pushbutton switch housing 218 which causes the sensor circuit to close, which in turn switches on the electronics unit as power from the battery is received.

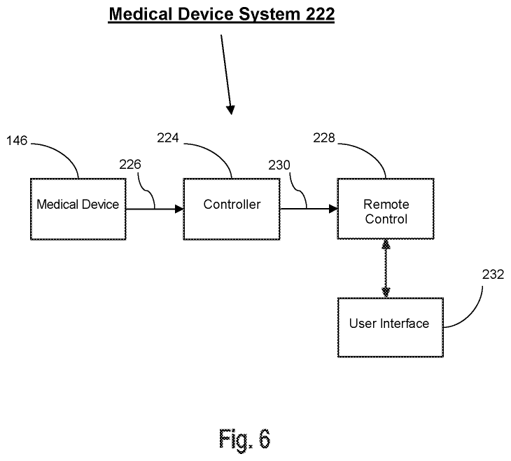

FIG. 6 shows an exemplary embodiment of a medical device system 222 according to this disclosure.

The medical device system 222 comprises a medical device 146. The medical device may correspond to the embodiments such as illustrated in FIGS. 2A and 2B, in FIGS. 3A and 3B or in FIGS. 4A to 4C. Thus, reference is made to the description of these figures above.

The medical device system 222 further comprises a sensor controller 224 which is coupled to the analyte sensor 110 of the medical device 146. The sensor controller is configured to receive analyte sensor data from the analyte sensor 110 such as indicated with arrow 226.

The medical device system 222 further comprises a remote control 228 which is configured to receive sensor data from the sensor controller 224, such as indicated with arrow 230, and to process and/or display sensor data such as via a user interface 232. The sensor controller 224 may be configured to communicate the sensor data to the remote control 228.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 analyte sensor
112 electrochemical sensor
114 in vivo proximal portion
115 insertable portion
116 ex vivo distal portion
118 Arrow
120 Carrier
122 Substrate
124 Electrode
125 electrode conductor path
126 first electrode
128 working electrode
130 second electrode
132 counter or reference electrode
134 contact pad
136 conductor path
138 contact pad
140 contact area
142 Surface
144 soldered contact element
146 medical device
147 insertion component
148 insertion cannula
150 Tip
152 slotted needle
154 housing
156 electronics compartment
158 upper side
160 lower side
162 distal side
164 proximal side
166 Surface
168 adhesive surface
170 open channel
172 upper side opening
174 lower side opening
176 open channel wall
178 first side
180 interior space
182 second side
184 interior space
186 removable upper cap
188 Holder
194 sensor compartment
200 connector element
202 contact spring element
204 bent contact spring element
206 leaf contact spring element
208 Surface
210 conductive elastomeric element
212 Surface
214 Protrusion
215 Arrow
216 pushbutton switch
218 pushbutton switch housing
220 movable element
222 medical device sensor system
224 sensor controller
226 Arrow
228 remote control
230 Arrow

What is claimed is:

1. A medical device for detecting an analyte in a body fluid, the medical device comprising:
an electronics unit having a sensor circuit;
an analyte sensor having an insertable portion adapted for at least partially being inserted into a body tissue of a user, the analyte sensor comprising (a) two conductor paths configured for activating the sensor circuit and (b) two electrodes each comprising an electrode conductor path configured for transmitting a sensor current for detecting the analyte;
an insertion cannula having the analyte sensor at least partially placed therein;
a housing having an electronics compartment with the electronics unit at least partially received therein and the housing having an open channel at least partially surrounding the analyte sensor and the cannula; and
a connector, wherein the insertion component is configured to retract from the open channel and thereby trigger electrical contact of the two conductor paths by the connector, which activates the sensor circuit.

2. The medical device according to claim 1, wherein the sensor circuit comprises an electrical energy reservoir and the connector is configured for establishing an electrical connection between the electrical energy reservoir and an electronic component of the sensor circuit by electrically contacting the two conductor paths of the analyte sensor.

3. The medical device according to claim 1, wherein, before retraction of the insertion cannula from the open channel, the sensor circuit is in an inactive state and wherein, during and/or after retraction of the insertion cannula from the open channel, the sensor circuit is in an active state.

4. The medical device according to claim 1, wherein the connector is configured for electrically contacting the two conductor paths of the analyte sensor by a movement, a displacement, a relaxation or an expansion of the connector or a component of the connector.

5. The medical device according to claim 1, wherein the connector is configured for establishing a transient or permanent electrical contact between the two conductor paths of the analyte sensor.

6. The medical device according to claim 1, wherein the housing comprises an upper side and a lower side and the open channel connects the upper side with the lower side, wherein the insertable portion of the analyte sensor extends downwardly in the open channel beyond the lower side of the housing.

7. The medical device according to claim 1, wherein each of the two conductor paths comprises a contact area, wherein the connector is configured for electrically contacting the contact areas of each of the two conductor paths.

8. The medical device according to claim 1, wherein the contact areas are disposed on a surface of the analyte sensor and are elevated relative to a surrounding surface of the analyte sensor.

9. The medical device according to claim 1, wherein the connector is selected from the group consisting of a contact spring and a conductive elastomer.

10. The medical device according to claim 9, wherein, before retraction of the insertion cannula from the open channel, the connector contacts a surface of the insertion cannula, further wherein, during and/or after retraction of the insertion cannula from the open channel, the connector electrically contacts the two conductor paths.

11. The medical device according to claim 1, wherein the connector is movably attached to a surface of the analyte sensor and the insertion cannula is configured for moving the connector to electrically contact the two conductor paths.

12. The medical device according to claim 1, wherein the open channel is formed by an open channel wall, wherein the connector is attached to a surface of the open channel wall facing an interior space enclosed by the open channel.

13. The medical device according to claim 12, wherein, before retraction of the insertion cannula from the open channel, the insertion cannula comprises a non-conductive spacer between the connector and the two conductor paths.

14. The medical device according to claim 1, further comprising:
a removable upper cap attached to the insertion cannula, wherein the removable upper cap is configured for removal after insertion of the insertable portion of the analyte sensor into the body tissue; and
a removable lower cap configured for removal before insertion of the insertable portion of the analyte sensor into the body tissue;
wherein the removable lower cap, the removable upper cap and the open channel form a sealed sensor compartment for at least partially receiving the analyte sensor.

15. The medical device according to claim 14, wherein at least one of the removable upper cap and the removable lower cap are removably connected to the housing and/or wherein the removable upper cap and the removable lower cap are removably connected to each other.

16. The medical device according to claim 15, wherein the removable upper cap seals with an upper side opening of the open channel and wherein the removable lower cap seals with a lower side opening of the open channel.

17. A medical device system, comprising:
a medical device according to claim 1;
a sensor controller coupled to the analyte sensor and configured to receive analyte sensor data from the analyte sensor; and
a remote control configured to receive sensor data from the sensor controller and to process and/or display sensor data.

18. A method of using the medical device according to claim 1, the method comprising:
a) providing the medical device;
b) inserting the analyte sensor into the body tissue; and
c) retracting the insertion cannula from the open channel, whereby an electrical contact of the two conductor paths of the analyte sensor by the connector is triggered to activate the sensor circuit.
